SEQUENCE LISTING (42 sequences)

SEQ ID NO: 1 - RNA, 21 nt, Artificial Sequence
  siRNA0097
uaaguucauguugaucugguu

SEQ ID NO: 2 - RNA, 21 nt, Artificial Sequence
  siRNA0145
uagcuugauguugaucugguu

SEQ ID NO: 3 - DNA, 97 bp, Artificial Sequence
  amiRNA0097
agctccttgttaagttcatgttgatctggcaagtctcttggatctcaaatgccactgaaccctttgccagatcaagttgaacttacaacacgggttt

SEQ ID NO: 4 - DNA, 97 bp, Artificial Sequence
  amiRNA0145
agctccttgttagcttgatgttgatctggcaagtctcttggatctcaaatgccactgaaccctttgccagatcaagttcaagctacaacacgggttt

SEQ ID NO: 5 - DNA, 10509 bp, Artificial Sequence
  pKS100 (amiRNA0097)
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgtacgtagcactagtgaattccggacccaagctttggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtggatctccaccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctgctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcggaccctcatgagcggagaattaagggagtcacgttatgacccccgccgatgacgcgggacaagccgttttacgtttggaactgacagaaccgcaacgaagctttggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtggatccagctccttgttaagttcatgttgatctggcaagtctcttggatctcaaatgccactgaaccctttgccagatcaagttgaacttacaacacgggtttgagctcttcatatgacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcggaccgaagcttgcatgcctgcaggtcgactctagaggatccggagccaagtctcataaacgccattgtggaagaaagtcttgagttggtggtaatgtaacagagtagtaagaacagagaagagagagagtgtgagatacatgaattgtcgggcaacaaaaatcctgaacatcttattttagcaaagagaaagagttccgagtctgtagcagaagagtgaggagaaatttaagctcttggacttgtgaattgttccgcctcttgaatacttcttcaatcctcatatattcttcttctatgttacctgaaaaccggcatttaatctcgcgggtttattccggttcaacattttttttgttttgagttattatctgggcttaataacgcaggcctgaaataaattcaaggcccaactgtttttttttttaagaagttgctgttaaaaaaaaaaaaagggaattaacaacaacaacaaaaaaagataaagaaaataataacaattactttaattgtagactaaaaaaacatagattttatcatgaaaaaaagagaaaagaaataaaaacttggatcaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttttttcttttctttattagagtagattagaatcttttatgccaagtattgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcaggtggatcccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatatgagtcgagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtaccatgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccccgggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 6 - DNA, 10509 bp, Artificial Sequence
  pKS101 (amiRNA0145)
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgtacgtagcactagtgaattccggacccaagctttggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtggatctccaccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctgctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcggaccctcatgagcggagaattaagggagtcacgttatgacccccgccgatgacgcgggacaagccgttttacgtttggaactgacagaaccgcaacgaagctttggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtggatccagctccttgttagcttgatgttgatctggcaagtctcttggatctcaaatgccactgaaccctttgccagatcaagttcaagctacaacacgggtttgagctcttcatatgacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcggaccgaagcttgcatgcctgcaggtcgactctagaggatccggagccaagtctcataaacgccattgtggaagaaagtcttgagttggtggtaatgtaacagagtagtaagaacagagaagagagagagtgtgagatacatgaattgtcgggcaacaaaaatcctgaacatcttattttagcaaagagaaagagttccgagtctgtagcagaagagtgaggagaaatttaagctcttggacttgtgaattgttccgcctcttgaatacttcttcaatcctcatatattcttcttctatgttacctgaaaaccggcatttaatctcgcgggtttattccggttcaacattttttttgttttgagttattatctgggcttaataacgcaggcctgaaataaattcaaggcccaactgtttttttttttaagaagttgctgttaaaaaaaaaaaaagggaattaacaacaacaacaaaaaaagataaagaaaataataacaattactttaattgtagactaaaaaaacatagattttatcatgaaaaaaagagaaaagaaataaaaacttggatcaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttttttcttttctttattagagtagattagaatcttttatgccaagtattgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcaggtggatcccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatatgagtcgagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtaccatgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccccgggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 7 - DNA, 12632 bp, Artificial Sequence
  Vector Control (15312-prCMP-GUS-tNOS)
aattcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgtacgtagcactagtgaattccggacccaagctttggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtggatctccaccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctgctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcggaccctcatgagcggagaattaagggagtcacgttatgacccccgccgatgacgcgggacaagccgttttacgtttggaactgacagaaccgcaacgaagctttggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtggatccccatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtaccaagctgcgaatcttcgtttttttaaggaattctcgatctttatggtgtataggctctgggttttctgttttttgtatctcttaggattttgtaaattccagatctttctatggccacttagtagtatatttcaaaaattctccaatcgagttcttcattcgcattttcagtcattttctcttcgacgttgtttttaagcctgggtattactcctatttagttgaactctgcagcaatcttagaaaattagggttttgaggtttcgatttctctaggtaaccgatctattgcattcatctgaatttctgcatatatgtcttagatttctgataagcttacgatacgttaggtgtaattgaagtttatttttcaagagtgttattttttgtttctgaatttttcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgagagctccgcggcgccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaccgaagcttgcatgcctgcaggtcgactctagaggatccggagccaagtctcataaacgccattgtggaagaaagtcttgagttggtggtaatgtaacagagtagtaagaacagagaagagagagagtgtgagatacatgaattgtcgggcaacaaaaatcctgaacatcttattttagcaaagagaaagagttccgagtctgtagcagaagagtgaggagaaatttaagctcttggacttgtgaattgttccgcctcttgaatacttcttcaatcctcatatattcttcttctatgttacctgaaaaccggcatttaatctcgcgggtttattccggttcaacattttttttgttttgagttattatctgggcttaataacgcaggcctgaaataaattcaaggcccaactgtttttttttttaagaagttgctgttaaaaaaaaaaaaagggaattaacaacaacaacaaaaaaagataaagaaaataataacaattactttaattgtagactaaaaaaacatagattttatcatgaaaaaaagagaaaagaaataaaaacttggatcaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttttttcttttctttattagagtagattagaatcttttatgccaagtattgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcaggtggatcccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatatgagtcgagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtaccatgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccccgggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatt

SEQ ID NO: 8 - DNA, 9634 bp, Artificial Sequence
  Vector Control (empty 15312)
aattcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgtacgtagcactagtgaattccggacccaagctttggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtggatctccaccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctgctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcggaccgaagcttgcatgcctgcaggtcgactctagaggatccggagccaagtctcataaacgccattgtggaagaaagtcttgagttggtggtaatgtaacagagtagtaagaacagagaagagagagagtgtgagatacatgaattgtcgggcaacaaaaatcctgaacatcttattttagcaaagagaaagagttccgagtctgtagcagaagagtgaggagaaatttaagctcttggacttgtgaattgttccgcctcttgaatacttcttcaatcctcatatattcttcttctatgttacctgaaaaccggcatttaatctcgcgggtttattccggttcaacattttttttgttttgagttattatctgggcttaataacgcaggcctgaaataaattcaaggcccaactgtttttttttttaagaagttgctgttaaaaaaaaaaaaagggaattaacaacaacaacaaaaaaagataaagaaaataataacaattactttaattgtagactaaaaaaacatagattttatcatgaaaaaaagagaaaagaaataaaaacttggatcaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttttttcttttctttattagagtagattagaatcttttatgccaagtattgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcaggtggatcccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatatgagtcgagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtaccatgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccccgggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatt

SEQ ID NO: 9 - RNA, 21 nt, Artificial Sequence
  siRNA with random seed sequence
unnnnnnnuguugaucugguu

SEQ ID NO: 10 - RNA, 21 nt, Artificial Sequence
  siRNA consensus sequence for random seed sequence
unnnnnnnuguugaucugguu

SEQ ID NO: 11 - RNA, 21 nt, Artificial Sequence
  siRNA consensus sequence for target-specific seed sequence
uarsuusauguugaucugguu

SEQ ID NO: 12 - RNA, 21 nt, Artificial Sequence
  siRNA
uaacuuauuguugaucugguu

SEQ ID NO: 13 - RNA, 21 nt, Artificial Sequence
  siRNA
uaacuucuuguugaucugguu

SEQ ID NO: 14 - RNA, 21 nt, Artificial Sequence
  siRNA
uagguugguguugaucugguu

SEQ ID NO: 15 - RNA, 21 nt, Artificial Sequence
  siRNA
uaucuucguguugaucugguu

SEQ ID NO: 16 - RNA, 21 nt, Artificial Sequence
  siRNA
ugacaggauguugaucugguu

SEQ ID NO: 17 - RNA, 21 nt, Artificial Sequence
  siRNA
ugaggucauguugaucugguu

SEQ ID NO: 18 - RNA, 21 nt, Artificial Sequence
  siRNA
ugguaugguguugaucugguu

SEQ ID NO: 19 - RNA, 21 nt, Artificial Sequence
  siRNA
ugggaucuuguugaucugguu

SEQ ID NO: 20 - RNA, 21 nt, Artificial Sequence
  siRNA
ugucagauuguugaucugguu

SEQ ID NO: 21 - RNA, 21 nt, Artificial Sequence
  siRNA
ugucgugauguugaucugguu

SEQ ID NO: 22 - RNA, 21 nt, Artificial Sequence
  siRNA
ugucuucguguugaucugguu

SEQ ID NO: 23 - RNA, 21 nt, Artificial Sequence
  siRNA
ugugugauuguugaucugguu

SEQ ID NO: 24 - DNA, 97 bp, Artificial Sequence
  amiRNA
agctccttgttaacttattgttgatctggcaagtctcttggatctcaaatgccactgaaccctttgccagatcaagtataagttacaacacgggttt

SEQ ID NO: 25 - DNA, 97 bp, Artificial Sequence
  amiRNA
agctccttgttaacttcttgttgatctggcaagtctcttggatctcaaatgccactgaaccctttgccagatcaagtagaagttacaacacgggttt

SEQ ID NO: 26 - DNA, 97 bp, Artificial Sequence
  amiRNA
agctccttgttaggttggtgttgatctggcaagtctcttggatctcaaatgccactgaaccctttgccagatcaagtccaacctacaacacgggttt

SEQ ID NO: 27 - DNA, 97 bp, Artificial Sequence
  amiRNA
agctccttgttatcttcgtgttgatctggcaagtctcttggatctcaaatgccactgaaccctttgccagatcaagtcgaagatacaacacgggttt

SEQ ID NO: 28 - DNA, 97 bp, Artificial Sequence
  amiRNA
agctccttgttgacaggatgttgatctggcaagtctcttggatctcaaatgccactgaaccctttgccagatcaagttcctgtcacaacacgggttt

SEQ ID NO: 29 - DNA, 97 bp, Artificial Sequence
  amiRNA
agctccttgttgaggtcatgttgatctggcaagtctcttggatctcaaatgccactgaaccctttgccagatcaagttgacctcacaacacgggttt

SEQ ID NO: 30 - DNA, 97 bp, Artificial Sequence
  amiRNA
agctccttgttggtatggtgttgatctggcaagtctcttggatctcaaatgccactgaaccctttgccagatcaagtccataccacaacacgggttt

SEQ ID NO: 31 - DNA, 97 bp, Artificial Sequence
  amiRNA
agctccttgttgggatcttgttgatctggcaagtctcttggatctcaaatgccactgaaccctttgccagatcaacaagatcccacaacacgggttt

SEQ ID NO: 32 - DNA, 97 bp, Artificial Sequence
  amiRNA
agctccttgttgtcagattgttgatctggcaagtctcttggatctcaaatgccactgaaccctttgccagatcaagtatctgacacaacacgggttt

SEQ ID NO: 33 - DNA, 97 bp, Artificial Sequence
  amiRNA
agctccttgttgtcgtgatgttgatctggcaagtctcttggatctcaaatgccactgaaccctttgccagatcaagttcacgacacaacacgggttt

SEQ ID NO: 34 - DNA, 97 bp, Artificial Sequence
  amiRNA
agctccttgttgtcttcgtgttgatctggcaagtctcttggatctcaaatgccactgaaccctttgccagatcaagtcgaagacacaacacgggttt

SEQ ID NO: 35 - DNA, 97 bp, Artificial Sequence
  amiRNA
agctccttgttgtgtgattgttgatctggcaagtctcttggatctcaaatgccactgaaccctttgccagatcaacaatcacacacaacacgggttt

SEQ ID NO: 36 - DNA, 348 bp, Glycine max
atggaatcctgcaattgtattgacccacaagtgccagctgatgacttattgatgaaataccaatatatctctgatttcttcattgcccttgcttatttctcaatccctttggagcttatctactttgttaagaaatcagcagtatttccatatagatgggtccttgttcagtttggtgcttttatagttttatgtggagcaacacatctaattaatttatggacctttagaattcattcaagaactgtggctgttgtaatgaccactgctaaggttttaactgctgtggtatcatgtgcaactgccctcatgctggtacatattattcctgatttattaagtgttaaa

SEQ ID NO: 37 - RNA, 20 nt, Glycine max
agaccgaucaaaugaauuua

SEQ ID NO: 38 - RNA, 20 nt, Glycine max
uggaaguggacuuggccugg

SEQ ID NO: 39 - RNA, 21 nt, Artificial Sequence
  amiRNA
ccagaucaaguugaacuuaca

SEQ ID NO: 40 - RNA, 21 nt, Artificial Sequence
  amiRNA
ccagaucaaguucaagcuaca

SEQ ID NO: 41 - RNA, 21 nt, Glycine max
ugcgaguacagguaaaagauu

SEQ ID NO: 42 - RNA, 25 nt, Glycine max
ggccugagguuucaacaagaaguua

(19) United States
(12) Patent Application Publication
(10) Pub. No.: US 2014/0047582 A1
(43) Pub. Date: Feb. 13, 2014

(54) INTERFERING RNAs THAT PROMOTE ROOT GROWTH

(71) Applicants: Xiang Huang, Research Triangle Park, NC (US); Thomas McNeill, Frisco, TX (US); Michael Schweiner, Research Triangle Park, NC (US); Peter Wittich, Research Triangle Park, NC (US)

(72) Inventors: Xiang Huang, Research Triangle Park, NC (US); Thomas McNeill, Frisco, TX (US); Michael Schweiner, Research Triangle Park, NC (US); Peter Wittich, Research Triangle Park, NC (US)

(73) Assignee: SYNGENTA PARTICIPATIONS AG, Basel (CH)

(21) Appl. No.: 13/985,440

(22) PCT Filed: Feb. 14, 2012
(86) PCT No.: PCT/US12/25012

§ 371 Date: Nov. 1, 2013

Related U.S. Application Data
(60) Provisional application No. 61/442,756, filed on Feb. 14, 2011.

(51) Int. Cl. C12N 15/113

(52) U.S. Cl. CPC C12N 15/113
USPC 800/285; 800/298

(57) ABSTRACT

siRNA, miRNA and amiRNA molecules having plant root growth promoting activity, compositions comprising the same, and uses thereof.

### FIELD OF THE INVENTION

The invention disclosed herein relates generally to compositions and methods for conferring tolerance to biotic and abiotic stress and for increasing yield in plants by modulating gene-specific silencing. More particularly, the invention relates to contacting a small interfering RNA molecule with a plant or plant part to thereby promote root growth in the plant.

### BACKGROUND

Many food sources are produced by crop plants. Environmental conditions such as drought and heat often adversely affect crop growth and yield. Pest pressure may also have a substantial negative impact. Consequently, plants that are capable of withstanding environmental stresses and/or pest challenge are desirable. Plants tolerant or resistant to abiotic and biotic stresses can be obtained by selective breeding or through genetic modification. RNA interference (RNAi) can be used to produce genetically modified plants that are tolerant or resistant to abiotic and biotic stresses.

In the past decade, RNAi has been described and characterized in organisms as diverse as plants, fungi, nematodes, hydra, and humans. Zamore and Haley (2005) Science 309, 1519-24. RNA interference in plants is commonly referred to as post-transcriptional gene silencing or RNA silencing and is referred to as quelling in fungi. The process of post-transcriptional gene silencing is thought to be an evolutionarily conserved cellular defense mechanism used to prevent the expression of foreign genes and is commonly shared by diverse flora and phyla. Fire (1999) Trends Genet. 15, 358-363.

RNA interference occurs when an organism recognizes double-stranded RNA molecules and hydrolyzes them. The resulting hydrolysis products are small RNA fragments of 19-24 nucleotides in length, called small interfering RNAs (siRNAs) or microRNAs (miRNAs). The interfering RNAs then diffuse or are carried throughout the organism, including across cellular membranes, where they hybridize to mRNAs (or other RNAs) and cause hydrolysis of the RNA. Most plant miRNAs show extensive base pairing to, and guide cleavage of their target mRNAs. Jones-Rhoades et al. (2006) Annu. Rev. Plant Biol. 57, 19-53; Llave et al. (2002) Proc. Natl. Acad. Sci. USA 97, 13401-10406. In other instances, interfering RNAs may bind to target RNA molecules having imperfect complementarity, causing translational repression without mRNA degradation.

There is a need to identify methods that promote tolerance or resistance to environmental challenges in crop plants, for example, by promoting morphological and/or biochemical changes in plants that enable improved responses to stress conditions. To meet this need, the invention described herein provides siRNA, miRNA and amiRNA compositions and methods that promote root growth.

### SUMMARY

The invention described herein provides compositions and methods for promoting root growth.

One aspect of the invention is a method for preparing a transgenic plant having a normal germination phenotype as compared to a control plant by expressing in a plant a nucleic acid a miRNA comprising a seed and a non-seed nucleic acid and having at least partial complementarity to a target nucleic acid and growing the transgenic plant having a normal germination phenotype. The target nucleic acid can be an ethylene response nucleic acid.

Another aspect of the invention is a method for preparing a transgenic plant wherein the seed nucleic acid is only partially complementary or fully complementary to the target nucleic acid. The target nucleic acid can be an ethylene response nucleic acid, an ETR1 nucleic acid, an ETR1 from soybean (Glycine max) or a nucleic acid have at least 60%, 70%, 80%, 90%, 95% or 100% identity to SEQ ID NO: 36. Alternatively, the miRNA comprises the seed sequence of SEQ ID NO: 1 or SEQ ID NO: 2.

Another aspect of the invention is a method for preparing a transgenic plant wherein the miRNA is contained within an amiRNA. The amiRNA may comprise a gma-MIR164 backbone and alternatively comprise SEQ ID NO: 3 or SEQ ID NO: 4. The miRNA may be designed so as the seed nucleic acid comprises nucleotides occupying positions 2-8 of the siRNA and wherein the non-seed nucleic acid comprises nucleotides occupying positions 1 and 9-21 of the siRNA. In addition, the seed nucleic acid may have 5, 6, or 7 nucleotides complementary to the target nucleic acid and be able to properly base pair with the target mRNA. Optionally, the amiRNA may be designed to produce two miRNAs that are at least partially complementary to an ethylene response mRNA.

Another aspect of the invention are methods for improving root growth in a plant while maintaining normal germination comprising transforming a plant cell with a vector comprising an amiRNA expressing a miRNA having at least partial complementarity to a plant ETR mRNA and regenerating a plant with improved root growth from the plant cell. The amiRNA may be operably linked to a tissue preferred promoter, a root preferred promoter or a CMP promoter.

Another aspect of the invention is use of a miRNA with at least partial complementarity to a plant ETR mRNA in a plant for improved root growth while maintaining normal germination. The miRNA may be expressed in a plant with a tissue preferred promoter, a root preferred promoter or a CMP promoter.

Another aspect is a plant produced by any of the methods or uses described previously.

### BRIEF DESCRIPTION OF THE SEQUENCES

The Sequence Listing provides disclosure of siRNAs, miRNAs and amiRNAs (artificial microRNAs) of the following sequences that are specific aspects of the invention.

SEQ ID NOs: 1-2, 12-23 are the nucleotide sequences of siRNAs, which are also listed in Table 1 and were used to design miRNAs contained within amiRNAs.

SEQ ID NOs: 3-6, 24-35 are the nucleotide sequences of amiRNAs, which are also listed in Table 2.

SEQ ID NO: 7 is the nucleotide sequence of 15312-prCMP-GUS-tNOS, which was used as a negative control in experiments to assess the activity of amiRNAs. See Example 2.

SEQ ID NO: 8 is the nucleotide sequence of the empty 15312 vector.

SEQ ID NO: 9 is the nucleotide sequence of a siRNA/miRNA containing a random seed sequence and a fixed non-seed sequence, as described in Example 1, below.

SEQ ID NO: 10 is the nucleotide sequence of a siRNA/miRNA containing a target-specific seed sequence and a fixed non-seed sequence, as described in Example 1, below.

SEQ ID NO: 11 is the nucleotide sequence comprising a consensus of siRNAs/miRNAs having plant root growth promoting activity, as described in Example 1, below.

SEQ ID NO: 36 is the nucleotide sequence of a soybean ETR1 nucleic acid (gma-ETR1), GenBank accession number EF210138.

SEQ ID NO: 37 is the nucleotide sequence comprising the mRNA portion of a soybean ETR1 that binds to the miRNA produced by amiRNA0097 and amiRNA0145.

SEQ ID NO: 38 is the nucleotide sequence comprising the mRNA portion of a soybean ETR1 that binds to the miRNA produced by amiRNA0097* and amiRNA0145*.

SEQ ID NO: 39 is the nucleotide sequence of the miRNA* produced by amiRNA0097.

SEQ ID NO: 40 is the nucleotide sequence of the miRNA* produced by amiRNA0145.

SEQ ID NO: 41 is the nucleotide sequence describing the mRNA sequence of a soybean ETR1 that does not effectively bind to the miRNA produced by amiRNA0043.

SEQ ID NO: 42 is the nucleotide sequence describing the mRNA sequence of a soybean ETR1 that does not effectively bind to the miRNA produced by amiRNA0046.

### DETAILED DESCRIPTION

The invention provides compositions and methods for conferring tolerance to biotic and abiotic stress and for increasing yields in plants by modulating gene-specific silencing. As described herein, the siRNAs are selected based upon an ability to promote root growth. In particular, siRNAs capable of modulating gene-specific silencing of ethylene response (ETR) nucleic acids, such as, ETR1, EIN1, QITR, Q8, TETR, TGETR1, TGETR2 and the like are capable of promoting root growth in plants. Please see, Negi, et al., The Plant Journal, vol. 55, pages 175-187 (2008) and U.S. Pat. No. 5,824,868. In addition, alteration in ethylene sensitivity in soybean has been implicated in pod retention and increased yield (for example, see U.S. Pat. No. 7,105,654).

As used herein, the phrases “promote root growth” or “promote root proliferation” refer to the ability of a siRNA or amiRNA to enhance development or expansion of roots from a plant or pant part. Root growth, as used herein, encompasses the gene expression, gene suppression, development, germination, differentiation, maturation, multiplication, amplification, enlargement, branching, plasticity, or gravitropism of any root, root tissues, or root structures. Growth encompasses primary growth from apical meristems and secondary growth from the vascular cambium, xylem, or phloem. Root growth can be an increase of root number, depth, length, diameter, branching, girth, mass, surface area, or the formation of rhizobia or mycorrhiza nodules, or other relevant measure.

Plants expressing siRNAs of the invention may also show increased yield, increased plant biomass, increased nutrient utilization (e.g., nitrogen use efficiency or increased nitrogen fixation), increased standability or resistance to wind stress, and other desirable agronomic indicators associated with increased root growth. Plants expressing siRNAs of the invention may also improve post-harvest soil conditions, such as increased nutrient retention and reduced soil erosion. These properties of the post-harvest land are beneficial to subsequent plantings, which require decreased supplementation of nutrients.

### Ethylene Reponses Nucleic Acids

A number of ethylene response nucleic acids are available for use in modulating ethylene response nucleic acids. Additional ethylene response nucleic acids can include, but are not limited to, ETR1, ETR2, EIN1, QITR, Q8, TETR, TGETR1, TGETR2 and the like. Please see, Negi, et al., The Plant Journal, vol. 55, pages 175-187 (2008).

For example, but in no means limiting, the ETR genes from Arabidopsis, as well as other plant homologues of ETR1 and ETR2, are considered to be ethylene receptors. (see, e.g., Gamble et al. (1998) PNAS USA 95, 7825-7829). The Arabidospsis ETR1 protein contains an amino-terminal half with a hydrophobic domain responsible for ethylene binding and membrane localization (Gamble et al. supra). The carboxyl-terminal half of the Arabidopsis ETR1 contains domains with homology to histidine kinases and response regulators (Gamble et al., supra).

Ethylene production in plants is involved in a plant's response to multiple biotic and abiotic stresses. Plants carrying mutations in ETR genes have been studied. For example, ethylene insensitive soybean plants with mutations in the ETR1 gene have been found to have increased resistance to some pathogens but reduced resistance to other pathogens (Hoffman et al., (1999) Plant Physiology 119, 935-949). Mutations in ethylene response genes in plants can cause other undesirable phenotypes. For example, strong ethylene insensitive mutants of soybean have extended hypocotyls during germination similar to an etiolated soybean seedling (Hoffman et al., supra). Extended hypocotyls during germination is an undesirable phenotype in a commercial soy variety. Ethylene insensitive lines of soybean demonstrate elevated susceptibility to white mold disease, altered plant architecture, including reduced seedling establishment, “flopping” petioles and earlier maturity date (Bent, et al., (2006) Crop Science, 46, 893-901). Therefore, controlled modulation of ethylene response gene expression in soybean is necessary for improved root growth and/or yield while still maintaining normal germination or other desirable phenotypes of a wild type plant or control plant. As used herein, “normal germination” or “having a normal germination phenotype”, means that a seed during germination does not have extended hypocotyls as compared to a wild type plant or control plant.

In addition, negative phenotypes resulting from knock out or mutation of an ethylene response gene or complete downregulation of an ethylene response gene in some or all of the tissues of a plant may be overcome by tissue preferred expression of an amiRNA. For example, root preferred expression of an amiRNA designed to downregulate an ethylene response gene, will reduce or eliminate the possible increase in disease susceptibility of pathogens that affect the leaves of a plant, such as soybean white mold (Sclerotinia stem rot). Alternatively, tissue preferred expression of an amiRNA in inflorescence, pod, ovule or seed may improve pod retention and/or yield, while minimizing negative effects. This can be independent from root enhancement or used in combination with root preferred expression of an amiRNA. It is possible to combine multiple constructs by either a molecular stack of two or more expression cassettes in a vector used for transforming a plant or one can combine traits through various breeding methods.

Another alternative could be tissue preferred expression of an amiRNA in the thin layer of cells of the abscission zone. In response to the hormone ethylene, abscission cells release cellulase and other enzymes that biochemically snip off flowers, leaves, fruit, stems, or seeds. Ethylene response genes send abscission orders when appropriate in response to various stimuli such as, the season, temperature, growth stage, disease or insect threats, abiotic stress or other events. See, for example, Tucker, et al., (1996) Agricultural Research 44(8), 8-10, for a discussion on ethylene regulation involved in abscission. By preventing an ethylene response in the abscission layer by tissue preferred expression of an amiRNA designed to downregulate an ethylene response gene, it is possible to prevent pod or seed abortion that may occur under abiotic stress and thereby increase yield in a plant, without incurring other negative side effects from general downregulation of an ethylene response gene.

Due to the nature of amiRNA regulation in attenuating rather than eliminating expression of a target gene, the combination of a tissue preferred promoter driving the expression of an amiRNA is able to attenuate the expression of an ethylene responsive gene in a plant to the appropriate level without negative phenotypes.

### Abiotic Stress

Increased abiotic stress tolerance and resistance, including enhanced drought tolerance, is correlated with increased root growth (see, e.g., Li et al. (2010) Plant, Cell and Environment 33, 272-289; Park et al. (2005) Proc. Natl. Acad. Sci. U.S.A. 102, 18830-18835; and Giuliani et al. (2005) Journal of Experimental Botany 56, 3061-3070). Accordingly, the disclosed methods for promoting root growth are useful for conferring increased abiotic stress tolerance or resistance.

As used herein, the phrases “abiotic stress,” “stress,” or “stress condition” refer to the exposure of a plant, plant part, plant cell, or the like, to a non-living, i.e., abiotic physical stress, chemical agents, or environmental conditions that can produce adverse effects on metabolism, growth, development, propagation, and/or survival of the plant (collectively “growth”). Abiotic stress can be imposed on a plant, for example, because of environmental factors such as water (e.g., flooding, drought, and dehydration), anaerobic conditions (e.g., a low level of oxygen), abnormal osmotic conditions, salinity or temperature (e.g., hot/heat, cold, freezing, frost), a deficiency of nutrients, exposure to pollutants, or by a exposure to hormone, second messenger or other molecule. Anaerobic stress, for example, is due to a reduction in oxygen levels (hypoxia or anoxia) sufficient to produce a stress response. A flooding stress can be due to prolonged or transient immersion of a plant, plant part, tissue, or isolated cell in a liquid medium such as occurs during a monsoon, wet season, flash flooding, or excessive irrigation of plants, or the like. A cold stress or heat stress can occur due to a decrease or increase, respectively, in the temperature from the optimum range of growth temperatures for a particular plant species. Such optimum growth temperature ranges are readily determined or known to those skilled in the art. Dehydration stress can be induced by the loss of water, reduced turgor, or reduced water content of a cell, tissue, organ, plant part, or whole plant. Drought stress can be induced by or associated with the deprivation of water or reduced supply of water to a cell, tissue, organ, or organism. Salinity-induced stress (i.e., salt-stress) can be associated with or induced by a perturbation in the osmotic potential of the intracellular or extracellular environment of a cell.

As used herein, “resistance to abiotic stress,” “abiotic stress resistance,” or “abiotic stress tolerance” includes, but is not limited to, greater water optimization; greater tolerance to dehydration, water deficit conditions, or drought; better recovery from dehydration, water deficit conditions, or drought; increased root growth; increased lateral root formation; increased root branching; increased surface area of roots; increased root mass; more root hairs; increased nutrient uptake; increased micronutrient uptake; increased metabolic efficiency; greater photosynthetic capacity; more rapid growth rate; greater fruit or seed yield; modified plant architecture; enhanced herbicide resistance; reduced or increased height; reduced or increased branching; enhanced cold and frost tolerance; improved vigor; enhanced color; enhanced health and nutritional characteristics; improved storage; enhanced yield; enhanced salt tolerance; enhanced resistance of wood or plant tissue to decay; enhanced heavy metal tolerance; enhanced sweetness; improved texture; decreased phosphate content; increased germination; improved starch composition; improved flower longevity; production of novel resins; production of novel proteins or peptides; enhanced agronomic traits, or any other agronomically desirable or commercially advantageous traits or characteristics.

As used herein, the phrase “enhanced abiotic stress tolerance” refers to a measurable improvement, enhancement, or increase in one or more of the above-identified traits as compared to one or more control plants (e.g., one or both of the parents, or a plant lacking a marker associated with enhanced abiotic stress tolerance).

As used herein, the phrase “water optimization” refers to any measure of a plant, its parts, or its structure that can be measured and/or quantified in order to assess an extent of or a rate of plant growth and development under different conditions of water availability. As such, a “water optimization trait” is any trait that can be shown to influence yield in a plant under different sets of growth conditions related to water availability. Exemplary measures of water optimization are grain yield at standard moisture percentage (YGSMN), grain moisture at harvest (GMSTP), grain weight per plot (GWTPN), and percent yield recovery (PYREC).

As used herein, the phrase “percent yield recovery” refers to an effect that a water optimization trait has when the trait is present in a plant on the yield of the plant under limited irrigation as compared to the yield under identical conditions that an otherwise genetically identical plant that lacks the trait would have. PYREC is calculated as:

$$1.00 - \frac{\begin{matrix}yield under full irrigation - \\ yield under limited irrigation with trait\end{matrix}}{\begin{matrix}yield under full irrigation - \\ yield under limited irrigation without trait\end{matrix}} \times 100$$

By way of example and not limitation, if a hybrid that yields 200 bushels in a full irrigation control treatment yields 100 bushels in a limited irrigation treatment, then the percentage yield loss would be calculated at 50%. If under the same conditions an otherwise genetically identical hybrid that contains the water optimization trait would attain a yield level of 125 bushes in the limited irrigation treatment, then the percentage yield loss would be calculated as 37.5% and the percentage yield recovery (PYREC) would be calculated as 25% (1.00−(200−125)/(200−100)×100).

Water optimization may also be described by expression of a “water use efficiency (WUE)” value. A water optimization trait may include improved yield under reduced or limited water availability, for example under drought conditions. As used herein, the phrases “drought tolerance” and “drought tolerant” refer to a plant's ability to endure and/or thrive under conditions where water availability is suboptimal. In general, a plant is labeled as “drought tolerant” if it displays “enhanced drought tolerance.”

As used herein, the phrase “enhanced drought tolerance” refers to a measurable improvement, enhancement, or increase in one or more water optimization phenotypes as compared to one or more control plants.

Plants expressing miRNAs of the invention, which show enhanced water optimization, also show one or more of enhanced drought tolerance, improved percent yield recovery or increased water optimization as compared to a control plant. In the case of crop plants, the enhanced water optimization trait may allow the crop to use less water during irrigation or allow the crop to be planted at a higher density when compared to control plants in a water limited environment. Plants expressing siRNAs of the invention, which show increased root growth, may also show a survival advantage in environments having a water surplus.

### Biotic Stress

The disclosed methods for promoting root growth are also useful for conferring tolerance to plant pests, particularly pests that initially attack a plant's root system, for example the corn rootworm (CRW) complex (Diabrotica spp.), which attacks the roots of corn plants. Newly hatched corn rootworms locate corn roots in the soil and initially begin feeding on the fine root hairs and burrow into root tips of the corn plant. As larvae grow larger, they feed on and tunnel into primary roots. When rootworms are abundant, larval feeding and deterioration of injured roots by root rot pathogens can result in roots being pruned to the base of the stalk. Severe root injury interferes with the roots' ability to transport water and nutrients into the plant, which reduces plant growth, and results in reduced grain production. Severe root injury also may result in lodging of corn plants, making harvest more difficult. Conversely, increased root mass and/or regrowth allow a plant to better tolerate rootworm feeding, with reduced likelihood of lodging. See e.g., Nowatzki et al. (2002) J. Econ. Entomol. 95: 570-577 and Marton et al. (2009) Maydica 54, 217-220.

As used herein, the phrase “biotic stress” refers to the exposure of a plant, plant part, plant cell, or the like, to a pest or pathogen that can produce adverse effects on metabolism, growth, development, propagation, and/or survival of the plant (collectively “growth”). Representative pests include nematodes and insects, and representative pathogens include fungi, bacteria, viruses, etc.

“Biotic stress tolerance” or “tolerance to a pest or pathogen,” as used herein, refers to the ability of a plant to withstand or reduce the severity of distress, infections, or disease caused by a pest or pathogen. “Enhanced tolerance to a pest or pathogen” refers to a measurable improvement, enhancement, or increase in one or more agronomic indicators as compared to a control plant (e.g., a plant lacking a miRNA associated with enhanced tolerance to a pest or pathogen). Tolerance can be measured by the plant's ability to survive infection by a pest or pathogen, reduced susceptibility to pest or pathogen infection, reduced pest or pathogen burden, increased yields, decreased attrition or death, or other suitable agronomic indicators, such as increased metabolic efficiency; greater photosynthetic capacity; more rapid growth rate; greater fruit or seed yield; modified plant architecture; reduced or increased height; reduced or increased branching; improved vigor; enhanced color; enhanced health and nutritional characteristics; enhanced yield; enhanced tolerance of wood or plant tissue to decay; enhanced heavy metal tolerance; improved texture; increased germination; improved flower longevity; or any other agronomically desirable or commercially advantageous traits or characteristics. Tolerance to a pest or pathogen can also be assessed by indicators of increased root growth, including for example, an increase of root number, depth, length, diameter, branching, girth, mass, surface area, or the formation of rhizobia or mycorrhiza nodules, or other relevant measure. In the context of pest pressure, “resistance to a pest or pathogen” indicates complete or substantially complete tolerance to a pest or pathogen.

In one aspect of the invention, siRNA/miRNAs molecules confer increased insect tolerance. Representative insect pests that may be targeted using siRNAs/miRNAs of the invention include without limitation chewing, sucking, and boring insects that belong, for example, to the Orders Coleoptera, Diptera, Hemiptera, Heteroptera, Homoptera, Hymenoptera, Lepidoptera, and Orthoptera. In a particular aspect of the invention, siRNA/miRNA molecules confer increased tolerance to corn rootworm. In another aspect of the invention, siRNA/miRNA molecules confer increased nematode tolerance.

### Interfering RNA Molecules

The invention provides siRNAs/miRNAs having plant root growth promoting activity. The scope of the invention is not limited to siRNAs/miRNAs for which specific sequences are disclosed herein. Rather, based upon the disclosure herein, one skilled in the art can readily design siRNAs/miRNAs having plant root growth promoting activity, for example, siRNAs/miRNAs comprising seed sequences of the disclosed siRNAs/miRNAs. In one embodiment, the siRNAs/miRNAs are directed to ETR nucleic acids. In particular, siRNAs/miRNAs directed to soybean ETR1 genes increase root growth promoting activity.

The term “RNA” includes any molecule comprising at least one ribonucleotide residue, including those possessing one or more natural ribonucleotides of the following bases: adenine, cytosine, guanine, and uracil; abbreviated A, C, G, and U, respectively, modified ribonucleotides, and non-ribonucleotides. “Ribonucleotide” means a nucleotide with a hydroxyl group at the 2′-position of the D-ribofuranose moiety. RNA can be single-stranded, double-stranded, isolated, partially purified, essentially pure, synthetic, recombinant, intracellular, and RNA that differs from naturally occurring RNA by the addition, deletion, substitution, and/or alteration of one or more nucleotides. “mRNA” refers to messenger RNA, which is RNA produced by transcription.

An “interfering RNA” (e.g., siRNA and miRNA) is a RNA molecule capable of post-transcriptional gene silencing or suppression, RNA silencing, and/or decreasing gene expression. Interfering RNAs effect sequence-specific, post-transcriptional gene silencing in animals and plants by base-pairing to the mRNA sequence of a target nucleic acid. Thus, the siRNA/miRNA is at least partially complementary to the silenced gene. The partially complementary siRNA/miRNA may include one or more mismatches, bulges, internal loops, and/or non-Watson-Crick base pairs (i.e., G-U wobble base pairs).

The terms “silencing” and “suppression” are used interchangeably to generally describe substantial and measurable reductions of the amount of mRNA available in the cell for binding and decoding by ribosomes. The transcribed RNA can be in the sense orientation to effect what is referred to as co-suppression, in the anti-sense orientation to effect what is referred to as anti-sense suppression, or in both orientations producing a double-stranded RNA to effect what is referred to as RNA interference. A “silenced” gene refers to a gene that is subject to silencing or suppression of the mRNA encoded by the gene.

The descriptions of microRNAs as “small interfering RNA”, “siRNA”, “miRNA” are used interchangeably herein to describe an interfering RNA. The term “amiRNA” refers to an artificial or non-natural microRNA comprising a miRNA contained within a pre-microRNA precursor. As used herein, “miRNA” refers to interfering RNAs that have been or will be processed in vitro or in vivo from a pre-microRNA precursor to form the active interfering RNA. Both siRNAs and miRNAs are RNA molecules of about 19-24 nucleotides, although shorter or longer siRNAs/miRNAs, e.g., between 18 and 26 nucleotides in length, may also be useful. siRNAs or miRNAs may be single stranded or double stranded. A siRNA molecule may be designed for increased stability if it is to be used as a composition to be sprayed on a plant or the like.

MicroRNAs are encoded by genes that are transcribed but not translated into protein (non-coding DNA), although some miRNAs are encoded by sequences that overlap protein-coding genes. miRNAs are processed from primary transcripts known as pri-miRNAs to short stem-loop structures called pre-miRNAs that are further processed creating functional siRNAs/miRNAs. Typically, a portion of the precursor miRNA is cleaved to produce the final miRNA molecule. The stem-loop structures may range from, for example, about 50 to about 80 nucleotides, or about 60 nucleotides to about 70 nucleotides (including the miRNA residues, those pairing to the miRNA, and any intervening segments). The secondary structure of the stem-loop structure is not fully base-paired; mismatches, bulges, internal loops, non-Watson-Crick base pairs (i.e., G-U wobble base pairs), and other features are frequently observed in pre-miRNAs and such characteristics are thought to be important for processing. Mature miRNA molecules are partially complementary to one or more messenger RNA molecules, and they function to regulate gene expression. siRNAs of the invention have structural and functional properties of miRNAs (e.g., gene silencing and suppressive functions). Thus, in various aspects of the invention, siRNAs of the invention can be processed from a portion of a precursor transcript that, optionally, folds into a stable hairpin (i.e., a duplex) or a stem-loop structure.

A target organism is an organism in which siRNAs/miRNAs of the invention are intended to be functional, i.e., to mediate gene silencing or suppression. In one aspect of the invention, a target organism is also a host organism (i.e., a target/host organism) as described herein below. In other aspects of the invention, a target organism is separate and distinct from a host organism that serves as a source of the siRNA/miRNAs to be functional in the target organism.

The terms “targeting” or “target(s),” as used herein, refer to the ability of siRNA/miRNAs molecules to form base pairs with a complementary mRNA molecule in a particular organism to thereby result in gene silencing or suppression. Such an organism is referred to as the target organism. A “target nucleic acid” or “target sequence” is a nucleic acid sequence or molecule from or in a target organism. “Target sequence” also implies a nucleic acid sequence that is selected for suppression and is not limited to polynucleotides encoding polypeptides. The target sequence typically comprises a sequence that is substantially or fully complementary to the siRNA/miRNA. The target sequence includes, but is not limited to, RNA, DNA, or other polynucleotide comprising the target sequence. In one embodiment the target sequence is a plant ethylene response nucleic acid. In another embodiment the target sequence is a plant ETR1. In another embodiment the target sequence is a soybean ETR1. In another embodiment the target sequence is SEQ ID NO: 36, a homologue of SEQ ID NO: 36 or a nucleic acid with at least 60%, 70%, 80%, 90%, 95% or 100% identity to SEQ ID NO: 36. Alternatively, the target sequence could be both a plant gene and a pest gene.

The invention provides target-specific siRNAs/miRNAs, i.e. siRNAs/miRNAs that selectively hybridize with nucleic acids in a target organism but not in a non-target organism, such as a consumer of the target organism. Consequently, “target-specific siRNAs” only produce a phenotype in target organisms and do not produce a phenotype in non-target organisms.

siRNAs/miRNAs of the invention may have multiple targets, for example, siRNAs/miRNAs may be functional in a plant as well as a plant pest. Representative plant pests and pathogens include fungi, nematodes, insects, bacteria, viruses, and parasitic plants such as striga, dodder, and mistletoe. Non-limiting examples of insects that may be targeted according to the invention include insects such as aphids, leafhoppers, planthoppers, mealy bugs, and Lepidoptera larvae.

A “host” is an organism that is intended for expression or production of a siRNA/miRNA. In one aspect of the invention, a host organism is the same as a target organism, i.e., the siRNA/miRNA is expressed in the same organism in which it is intended to be functional (referred to as a target/host herein). In another aspect of the invention, the host organism serves as a carrier of the siRNA/miRNA to a target organism. For example, a host organism is a plant, which may also be a target organism, and wherein a second target organism is a pest or pathogen of the plant.

In one aspect of the invention, a siRNA/miRNA molecule comprises (a) a seed sequence comprising nucleotides representative of one or more microRNA seed sequences of a target organism, an organism related to the target organism, or a consensus thereof and (b) a non-seed sequence comprising designated nucleotides. Such siRNA/miRNA molecules comprise at least about 17-19 nucleotides, wherein the seed sequence comprises 6-7 nucleotides.

The description “seed sequence” or “seed nucleic acid” as used herein, refers to a region of a siRNA or miRNA molecule that is at least partially complementary to a target gene or RNA. As used herein, the seed sequence consists of 6-7 nucleotides beginning at the second residue from the 5′-end of a siRNA/miRNA (e.g., nucleotides 2-7 or 2-8, as numbered linearly from the 5′-end of a siRNA or miRNA). A seed nucleic acid may have 1, 2, 3, 4, 5, 6, or 7 nucleotides that are complementary to a target nucleic acid. The seed sequences are the most highly conserved regions among metazoan miRNAs, and miRNAs with the same sequence at nucleotides 2-8 share the same predicted mRNA targets. See Bartel (2009) Cell 136, 215-233. A seed sequence may be designed to target a plant ethylene response nucleic acid, such as, a soybean ETR1 nucleic acid or a maize ETR1 nucleic acid. While seed and non-seed sequences do not need to be fully complementary, the design of the seed and non-seed sequences must still allow for proper base pairing with the target sequence. For example, miRNA0145 is partially complementary to a soybean ETR1 mRNA and allows for proper base pairing. On the other hand, miRNA0043 is partially complementary to a soybean ETR1 mRNA but does not allow for proper base pairing. Please see Example 1.

In one aspect of the invention, nucleotides within the seed sequence are based upon the frequency at which particular nucleotides are observed in miRNA seed sequences of a target organism, i.e., an organism in which a siRNA/miRNA of the invention is intended to be functional for gene silencing. Similarly, nucleotides within the seed sequence may be based upon the frequency at which particular nucleotides are observed in miRNA seed sequences of an organism related to the target organism. In this context, “related” means relative phylogenic closeness between and among organisms, whether evolutionary relationships are determined by phenotypic traits, molecular markers, and/or variation in rates of speciation and/or extinction. The degree of relation may be in some aspects, closely related through phylogeny, such as sharing the same genus or family. In other aspects, the degree of phylogenic relation may be distant, such as sharing only the same phylum or class. In other aspects, there may be no phylogenic relation to target an organism but the seed sequence may be related to the target organism through sequence homology, similarity, or identity. The consensus non-seed sequence can also be prepared from non-seed sequences from the target organism and/or from one or more organisms related to the target organism.

For example, the nucleotides of the seed sequence may be selected based upon observed frequencies at each position in naturally occurring miRNAs, for example, by excluding those nucleotides which are observed at a low frequency. A low frequency can comprise an observed incidence of less than about 50% among a population of naturally occurring miRNAs, or less than about 45%, or less than about 40%, or less than about 35%, or less than about 30%, or less than about 25%, or less than about 20%, or less than about 15%, or less than about 10%, or less than about 5%.

The seed sequence may alternatively comprise a consensus of two or more miRNA seed sequences of a target organism and/or an organism related to the target organism. The phrase “consensus sequence,” as used herein, refers to a nucleotide sequence wherein each nucleotide represents the most frequently observed nucleotide at a particular position in the sequence when similar or related sequences are compared to each other as described herein for determining the similarity or identity (see below). As used herein, the consensus sequence of a siRNA/miRNA, or part thereof, refers to either a selected group of siRNAs/miRNAs or all siRNAs/miRNAs that are conserved within an organism, species, genus, family, order, class, phylum, kingdom, or domain. The term “consensus” also encompasses structural elements known or predicted from the sequence, or from analogous or homologous sequences, such as duplexes, mismatches, budges, G-U wobble base pairs, loops, hairpins, tetraloops, inter alia, which are observed in pri-mRNA, pre-miRNA, miRNA, or siRNA sequences that are thought to be important for miRNA processing. See, e.g., Saxena et al. (2003) J. Biol. Chem. 278, 44312-44319.

Representative siRNA/miRNA seed sequences of the invention include residues 2-8 of any one of SEQ ID NOs: 1, 2, 10, 11, 12-23, 39 and 40.

The description “non-seed sequence,” as used herein, refers to all nucleotides of a siRNA or miRNA that are not the seed sequence. For a 21-nucleotide siRNA or miRNA, the non-seed sequence comprises linear nucleotides 1 and 8-21 or 1 and 9-21, depending on whether the seed sequence consists of 6 nucleotides (e.g., positions 2-7) or 7 nucleotides (e.g., positions 2-8). In one aspect of the invention, the non-seed sequence comprises a naturally occurring miRNA non-seed sequence. In another aspect of the invention, the non-seed sequence comprises a consensus microRNA non-seed sequence, i.e., a consensus of miRNA non-seed sequences. Such a consensus may be prepared from two or more miRNA non-seed sequences, for example, three miRNA sequences, or four miRNA sequences, or five miRNA sequences, or six miRNA sequences, or seven miRNA sequences, or eight miRNA sequences, or nine miRNA sequences, or ten miRNA sequences, or twenty miRNA sequences, or thirty miRNA sequences, or forty miRNA sequences, or fifty miRNA sequences, or more. One skilled in the art understands techniques and computational tools for making such alignments and can readily prepare consensus sequences using any number of miRNA sequences.

In one aspect of the invention, the miRNA non-seed sequence or consensus of miRNA non-seed sequences comprises a consensus of non-seed sequences from a target organism, i.e., an organism in which a siRNA/miRNA of the invention is intended to be functional for gene silencing. Similarly, the consensus of miRNA non-seed sequences can comprise a consensus of non-seed sequences related to the target organism. In this context, “related” means relative phylogenic closeness between or among organisms, as described herein with respect to design of seed sequences.

In another aspect of the invention, the non-seed sequence is partially or completely synthetic, i.e., a non-naturally occurring sequence that shows desired functional properties as determined by modeling or empirically. For example, the non-seed sequence can comprise one or more nucleotide substitutions relative to a naturally occurring miRNA sequence, a siRNA sequence, or a miRNA/siRNA consensus sequence to improve siRNA duplex stability, such as 3′-terminal uridines or deoxythymidine.

Where the target organism is a plant, a useful non-seed sequence can comprise a consensus of miRNA non-seed sequences of the model plant Arabidopsis thaliana. As another example, other useful seed sequences include consensus sequences of miRNA non-seed sequences of one or more other known plants, including any combination of target/host plants identified in herein below. A non-seed sequence may comprise a sequence with homology or complementarity to an ethylene response nucleic acid, such as, an ETR1 nucleic acid. A representative non-seed sequence is set forth as nucleotides 1 and 9-21 of SEQ ID NO: 9.

The invention also provides “artificial microRNAs” or “amiRNAs,” which are non-naturally occurring nucleic acid sequences that are capable of expressing miRNA or siRNA molecules. In one aspect of the invention, the sequence of the Glycine max miRNA precursor gma-MIR164 was used as the starting sequence or backbone for designing an artificial microRNA that will be expressed in a plant host. The design of this artificial microRNA for use in soybeans is described in U.S. Provisional Application 61/421,275 and a similar approach for use of amiRNAs in Arabidopsis thaliana is described by Schwab et al. (2006) Plant Cell 18, 1121-1133, both of which are incorporated herein by reference in their entirety. Representative amiRNAs of the invention include amiRNAs comprising a siRNA of SEQ ID NO: 1, 12-23, for example, the amiRNAs set forth as SEQ ID NOs: 3-6 and 24-35.

The above-described siRNAs/miRNA, or seed or non-seed sequences therein, or precursors thereof (e.g., pri-miRNA and pre-miRNA), may be further altered by the addition, deletion, substitution, and/or alteration of one or more nucleotides to introduce variation; to modify specificity; to alter complementarity; to introduce or remove secondary structural elements such as mismatches, bulges, loops, single-stranded regions, double-stranded regions, overhangs, or other motifs; to enhance or maintain the capability of the RNA to be processed in a RISC complex in vitro or in vivo; to improve the stability of the RNA molecule in vitro or in vivo (i.e., the ability of the RNA molecule to be maintained without being degraded by nucleases and/or its ability to fold into stable secondary or tertiary structures); and/or to enhance the ability to hybridize to a target gene/RNA.

Nucleic acids that share a substantial degree of complementarity will form stable interactions with each other, for example, by matching base pairs. The terms “complementary or “complementarity” refer to the specific base pairing of nucleotide bases in nucleic acids. The phrase “perfect complementarity,” as used herein, refers to complete (100%) base paring within a contiguous region of nucleic acid, such as between a seed sequence in a siRNA/miRNA and its complementary sequence in a target gene/RNA, as described herein. “Partial complementarity” or “partially complementary” indicates that two sequences can base pair with one another, although the complementarity is not 100%. As used herein, the phrase “sequence complementary to a sequence” is used to describe a nucleotide sequence capable of base pairing with another sequence, although the complementarity may not be 100%.

Alternatively stated, the phrase “sequence complementary to a sequence” or “nucleic acid complementary to a nucleic acid” with respect to two nucleotide sequences or nucleic acids indicates that the two-nucleotide sequences or nucleic acids have sufficient complementarity and have the natural tendency to interact with each other to form a double stranded molecule. Two nucleotide sequences or nucleic acids can form stable interactions with each other within a wide range of sequence complementarities. Nucleotide sequences or nucleic acids with high degrees of complementarity are generally stronger and/or more stable than ones with low degrees of complementarity. Different strengths of interactions may be required for different processes. For example, the strength of interaction for the purpose of forming a stable nucleic acid duplex in vitro may be different from that for the purpose of forming a stable interaction between a siRNA/miRNA and a binding nucleic acid in vivo. The strength of interaction can be readily determined experimentally or predicted with appropriate software by a person skilled in the art.

When an amiRNA comprising one of the above-described siRNAs or miRNAs, or seed and non-seed sequences/nucleic acids therein or precursors thereof is processed in a RISC complex in vitro or in vivo, the siRNA or miRNA may be designed to use both strands of the double stranded regions to deliver two siRNAs or miRNAs to the RISC complex. The amiRNAs comprising two siRNAs or miRNAs can be designed to bind to one target within a particular nucleic acid, as well as, a second target within the same or different nucleic acid. For example, amiRNA0097 and amiRNA0147 are processed in a plant to produce two miRNAs that can bind two different targets within the soybean ETR1 nucleic acid. In addition, the amiRNA may be designed to deliver one or more miRNAs that bind both a plant target nucleic acid and a pest target nucleic acid. Alternatively the amiRNA may be designed to deliver two siRNAs or miRNAs to bind to one or more plant and/or pest target nucleic acids.

The terms “hybridize” or “hybridization,” as used herein, refers to the ability of a nucleic acid sequence or molecule to base pair with a complementary sequence and form a duplex nucleic acid structure. Hybridization can be used to test whether two polynucleotides are substantially complementary to each other and to measure how stable the interaction is. Polynucleotides that share a sufficient degree of complementarity will hybridize to each other under various hybridization conditions. Consequently, polynucleotides that share a high degree of complementarity thus form strong stable interactions and will hybridize to each other under stringent hybridization conditions. “Stringent hybridization conditions” are well known in the art, as described in Sambrook et al. (1989) Molecular Cloning: A Laboratory Manual, Second Edition, Cold Spring Harbor Laboratory, Cold Spring Harbor, N.Y. An exemplary stringent hybridization condition comprises hybridization in 6× sodium chloride/sodium citrate (SSC) at about 45° C., followed by one or more washes in 0.2×SSC and 0.1% SDS at 50-65° C.

“Homologous,” “homology,” “identical,” and “identity,” as used herein, refers to comparisons among nucleic acid sequences. When referring to nucleic acid molecules, “homology,” “similarity,” “identity,” or “percent identity,” refers to the percentage of the nucleotides of a particular nucleic acid sequence that have been matched to similar or identical nucleotide sequences by a sequence analysis program. Sequence “identity” or “similarity,” as known in the art, is the relationship between two or more polynucleotide sequences, as determined by comparing the sequences. In the art, identity also means the degree of sequence relatedness between polynucleotide sequences, as determined by the match between such sequences. To determine the percent identity or similarity of two nucleic acid sequences, the sequences are aligned for optimal comparison purposes (i.e., gaps can be introduced in the sequence of a first nucleic acid sequence for optimal alignment with a second nucleic acid sequence). The nucleotides at corresponding nucleotide positions are then compared. When a position in the first sequence is occupied by the same or similar nucleotide as the corresponding position in the second sequence, then the molecules are identical or similar at that position, respectively. The percent identity or similarity between the two sequences is a function of the number of identical or similar positions shared by the sequences (i.e., the percentage (%) identity is number of identical positions divided by the total number of positions (e.g., overlapping positions)×100). Two sequences that share 100% sequence identity are identical. Two sequences that share less than 100% identity, but greater than 50% identity, are similar. Sequences with less than 50% identity are dissimilar. Sequences can share 50%, 60%, 70%, 75%, 80%, 85%, 90%, 95%, 96%, 97%, 98%, 99%, 100% or any integer of %.

Both identity and similarity can be readily calculated. Methods commonly employed to determine identity or similarity between or among sequences include, but are not limited to, those disclosed in Carillo et al. (1988) SIAM J. Applied Math. 48, 1073. A non-limiting example of a mathematical algorithm utilized for the comparison of two sequences is the algorithm of Karlin et al. (1990) Proc. Natl. Acad. Sci. USA 87, 2264-2268, modified as in Karlin et al. (1993) Proc. Natl. Acad. Sci. USA 90, 5873-5877. Such an algorithm is incorporated into the NBLAST and XBLAST programs of Altschul et al. (1990) J. Mol. Biol. 215, 403-410. To obtain gapped alignments for comparison purposes, Gapped BLAST can be utilized as described in Altschul et al. (1997) Nucleic Acids Res., 25, 3389-3402. Alternatively, PSI-Blast can be used to perform an iterated search that detects distant relationships between molecules. When utilizing BLAST, Gapped BLAST, and PSI-Blast programs, the default parameters of the respective programs (e.g., XBLAST and NBLAST) can be used. Additionally, the FASTA method can also be used. See Atschul et al. (1990) J. Mol. Biol. 215, 403-410. Another example of a mathematical algorithm useful for the comparison of sequences is the algorithm of Myers et al. (1988) CABIOS 4, 11-17. The percent identity between two sequences can also determined using the algorithm of Needleman and Wunsch (1970) J. Mol. Biol. 48, 444-453. Another algorithm for calculating the percent identity between two sequences is determined using the local homology method. Smith and Waterman (1981) J. Mol. Biol., 147, 195-197. Optimal alignments may be produced by inserting gaps to maximize the number of matches.

The invention provides methods for attenuating or inhibiting gene expression in a cell using small interfering RNA. The miRNA contains a nucleotide sequence that hybridizes under physiologic conditions of the cell to the nucleotide sequence of at least a portion of the target mRNA of the gene to be inhibited (i.e., the target gene). The methods described herein do not require 100% sequence identity or complementarity between the siRNA/miRNA and the target gene. By utilizing bioinformatic tools, the sequence can contain mismatching pairs of nucleotides. Thus, the methods of the invention have the advantage of being able to tolerate some sequence variations that might be expected due to genetic mutation, strain polymorphism, or evolutionary divergence.

Without being bound by theory, it is believed that plants transformed according to the invention transcribe an RNA molecule(s) with a region homologous to and a region complementary to the target gene, including a pest target gene, and wherein the transcript(s) form a double stranded RNA molecule (dsRNA). The plant recognizes the dsRNA as a potential foreign substance (e.g., a substance of viral origin). The dicer enzyme of the plant cuts the double stranded RNA into pieces of single-stranded RNA of about 23 nucleotides in length, called small interfering RNAs. These small interfering RNAs may be consumed by invading pests that have entered the plant via the digestion of plant cells (e.g., cutin), as well as, alter the expression of a plant gene. Once absorbed by the pest, the small interfering RNAs can be incorporated into the pest's RNA-induced silencing complexes. The RISC complex can then digest the mRNA of the pest's homologous gene limiting the pest's ability to harm the plant.

### Compositions Comprising Interfering RNAs

The invention also provides nucleic acids comprising the disclosed siRNAs, miRNAs, artificial miRNAs, and siRNA libraries. Such nucleic acids are generally useful for expression of the siRNAs/miRNAs in a manner that they can come into contact with target nucleic acids, i.e., nucleic acids to be regulated by the siRNA/miRNAs.

In the context of the invention, the phrase “nucleic acid” refers to oligonucleotides and polynucleotides such as ribonucleic acid (RNA) and deoxyribonucleic acid (DNA). The phrase nucleic acid should also be understood to include, as applicable, single-stranded (such as sense or antisense) and double-stranded polynucleotides. Nucleic acids according to the invention may be partially or wholly synthetic, and may be isolated and/or purified (i.e., from their natural environment), in substantially pure or homogeneous form, or free or substantially free of other nucleic acid.

Representative nucleic acids comprising siRNAs/miRNAs of the invention include expression constructs and vectors. The phrase “expression construct” refers to a nucleic acid suitable for expression in a cell. The term “vector” refers to a nucleic acid molecule (plasmid, virus, bacteriophage, artificial, heterologous, or cut DNA molecule) that can be used to deliver a heterologous or natural polynucleotide of the invention into a host cell. Vectors are capable of being replicated and contain cloning sites for introduction of a foreign polynucleotide.

Those skilled in the art are readily able to prepare expression constructs and vectors of the invention and recombinantly express the same. For further details see, e.g., Sambrook et al. (1989) Molecular Cloning: A Laboratory Manual, Second Edition, Cold Spring Harbor Laboratory, Cold Spring Harbor, N.Y. Such applicable techniques and protocols for manipulation of nucleic acid, for example in preparation of nucleic acid constructs, mutagenesis, sequencing, introduction of DNA into cells and gene expression, and analysis of proteins, are described in detail in Protocols in Molecular Biology, Second Edition, Ausubel et al. eds., John Wiley & Sons, 1992.

Specific expression techniques and vectors previously used with wide success upon plants are described by Bevan, Nucl. Acids Res. (1984) 12, 8711-8721, and Guerineau and Mullineaux, (1993) “Plant transformation and expression vectors,” Plant Molecular Biology Labfax (Croy RRD, ed.) Oxford, BIOS Scientific Publishers, 121-148.

Expression constructs include a promoter operably linked to a nucleic acid comprising a siRNA or miRNA, for example, an artificial microRNA, as described herein. Useful promoters include constitutive promoters, promoters that direct spatially and temporally regulated expression (e.g., tissue-preferred and developmental stage-preferred promoters), and inducible promoters. Expression constructs may also include enhancers of gene expression as known in the art.

Tissue-preferred promoters can be utilized to target enhanced expression of a sequence of interest within a particular plant tissue. Tissue-preferred promoters include Yamamoto et al. (1997) Plant J. 12, 255-265; Kawamata et al. (1997) Plant Cell Physiol. 38, 792-803; Hansen et al. (1997) Mol. Gen. Genet. 254, 337-343; Russell et al. (1997) Transgenic Res. 6, 157-168; Rinehart et al. (1996) Plant Physiol. 112, 1331-1041; Van Camp et al. (1996) Plant Physiol. 112, 525-535; Canevascini et al. (1996) Plant Physiol. 112, 513-524; Yamamoto et al. (1994) Plant Cell Physiol. 35, 773-778; Lam (1994) Results Probl. Cell Differ. 20, 181-196; Orozco et al. (1993) Plant Mol. Biol. 23, 1129-1138; Matsuoka et al. (1993) Proc Natl. Acad. Sci. USA 90, 9586-9590; and Guevara-Garcia et al. (1993) Plant J. 4, 495-505. Such promoters can be modified, if necessary, for weak expression.

In one aspect of the invention, siRNAs/miRNAs having root growth promoting activity are selectively expressed in target/host plant roots. Root-preferred promoters are known and can be selected from the many available from the literature or isolated de novo from various compatible species. See, e.g., Hire et al. (1992) Plant Mol. Biol. 20, 207-218 (soybean root-specific glutamine synthetase gene); Keller and Baumgartner (1991) Plant Cell 3, 1051-1061 (root specific control element in the GRP 1.8 gene of French bean); Sanger et al. (1990) Plant Mol. Biol. 14, 433-443 (root specific promoter of the mannopine synthase (MAS) gene of Agrobacterium tumefaciens); Stavolone et al. (2003) Journal of General Virology 84, 3459-3464 (CMP promoter) and Miao et al. (1991) Plant Cell 3, 11-22 (i.e., a full-length cDNA clone encoding cytosolic glutamine synthetase (GS), which is expressed in roots and root nodules of soybean). See also Bogusz et al. (1990) Plant Cell 2, 633-641, where two root-specific promoters isolated from hemoglobin genes from the nitrogen-fixing non legume Parasponia andersonii and the related non-nitrogen fixing non legume Trema tomentosa are described. The promoters of these genes were linked to a 13-glucuronidase reporter gene and introduced into both the non-legume Nicotiana tabacum and the legume Lotus corniculatus, and in both instances root-specific promoter activity was preserved. Leach and Aoyagi (1991) describe their analysis of the promoters of the highly expressed roIC and rolD root-inducing genes of Agrobacterium rhizogenes. See Plant Science (Limerick) 79, 69-76. They concluded that enhancer and tissue-preferred DNA determinants are dissociated in those promoters. Teen et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene. See EMBO J. 8 343-350. The TR1′ gene, fused to nptII (neomycin phosphotransferase II) showed similar characteristics. Additional root-preferred promoters include the VfENOD-GRP3 gene promoter and rolB promoter. See also Kuster et al. (1995) Plant Mol. Biol. 29, 759-772; Capana et al. (1994) Plant Mol. Biol. 25, 681-691. See also U.S. Pat. Nos. 5,837,876; 5,750,386; 5,633,363; 5,459, 252; 5,401,836; 5,110,732; and 5,023,179. The phaseolin gene is described by Murai et al. (1983) Science 23, 476-482, and Sengopta-Gopalen et al. (1988) Proc. Natl. Acad. Sci. USA 82, 3320-3324.

In other aspects of the invention, leaf-preferred promoters are known in the art. See, e.g., Yamamoto et al. (1997) Plant J. 12, 255-265; Kwon et al. (1994) Plant Physiol. 105, 357-67; Yamamoto et al. (1994) Plant Cell Physiol. 35, 773-778; Gotor et al. (1993) Plant J. 3, 509-18; Orozco et al. (1993) Plant Mol. Biol. 23, 1129-1138; and Matsuoka et al. (1993) Proc. Natl. Acad. Sci. USA 90, 9586-9590. In addition, the promoters of cab and rubisco can also be used. See, e.g., Simpson et al. (1958) EMBO J. 4, 2723-2729 and Timko et al. (1988) Nature 318, 57-58.

In some aspects, it will be beneficial to express miRNAs/siRNAs of the invention using an inducible promoter, such as from a pest or pathogen-inducible promoter. Such promoters include those from pathogenesis-related proteins (PR proteins), which are induced following infection by a pathogen; e.g., PR proteins, SAR proteins, β-1,3-glucanase, chitinase, etc. See, e.g., Redolfi et al. (1983) Neth. J. Plant Pathol. 89:245-254; Uknes et al. (1992) Plant Cell 4, 645-656; and Van Loon (1985) Plant Mol. Virol. 4, 111-116. See also PCT International Publication No. WO 99/43819.

Where a siRNA/miRNA has activity in enhancing abiotic resistance as well as activity in enhancing pest tolerance, promoters that are expressed locally at or near the site of pest or pathogen infection may be used. See, e.g., Marineau et al. (1987) Plant Mol. Biol. 9, 335-342; Matton et al. (1989) Molecular Plant-Microbe Interactions 2, 325-331; Somsisch et al. (1986) Proc. Natl. Acad. Sci. USA 83, 2427-2430; Somsisch et al. (1988) Mol. Gen. Genet. 2, 93-98; and Yang (1996) Proc. Natl. Acad. Sci. USA 93, 14972-14977. See also, Chen et al. (1996) Plant J. 10, 955-966; Zhang et al. (1994) Proc. Natl. Acad. Sci. USA 91, 2507-2511; Warner et al. (1993) Plant J. 3, 191-201; Siebertz et al. (1989) Plant Cell 1, 961-968; U.S. Pat. No. 5,750,386 (nematode-inducible); Cordero et al. (1992) Physiol. Mol. Plant. Path. 41, 189-200, and the references cited therein.

Additionally, as pathogens enter host plants through wounds or insect damage, a wound-inducible promoter may be used in the constructs of the invention. Such wound-inducible promoters include potato proteinase inhibitor (pin II) gene (Ryan (1990) Ann. Rev. Phytopath. 28, 425-449; Duan et al. (1996) Nature Biotech. 14, 494-498); wun1 and wun2, U.S. Pat. No. 5,428,148; winl and wing (Stanford et al. (1989) Mol. Gen. Genet. 215, 200-208); systemin (McGurl et al. (1992) Science 225, 1570-1573); WIP1 (Rohmeier et al. (1993) Plant Mol. Biol. 22, 783-792; Eckelkamp et al. (1993) FEBS Lett. 323, 73-76); and the MPI gene (Corderok et al. (1994) Plant J. 6, 141-150).

U.S. Pat. Nos. 5,589,622 and 5,824,876 describe the identification of plant genes expressed specifically in or adjacent to the feeding site of the plant after attachment by a nematode. The promoters of these plant target genes can then be used to direct the specific expression of detrimental amiRNA to the pest target gene.

In addition to the above-identified promoters, U.S. Patent Application Publication Numbers 2004/0016025, 2007/0056055, 2008/0120750, 2009/0183283, and U.S. Pat. Nos. 7,550,578 and 7,615,624 describe a variety of promoters from Oryza sativa and Arabidopsis thaliana, which may also be used for expression of siRNAs/miRNAs as described herein. The particular promoter sequences of the just named patent documents, and disclosure regarding use of such promoters, are incorporated by reference herein.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical inducible promoters are known in the art and include, but are not limited to, the maize Int-2 promoter, which is activated by benzenesulfonamide herbicide safeners, the maize GST promoter, which is activated by hydrophobic electrophilic compounds that are used as pre-emergent herbicides, and the tobacco PR-1 a promoter, which is activated by salicylic acid. Other chemical-regulated promoters of interest include steroid steroid-responsive promoters (see, e.g., the glucocorticoid-inducible promoter in Schena et al. (1991) Proc. Natl. Acad. Sci. USA 88, 10421-10425 and McNellis et al. (1998) Plant J. 14, 247-257) and tetracycline-inducible and tetracycline-repressible promoters (see, e.g., Gatz et al. (1991) Mol. Gen. Genet. 227, 229-237, and U.S. Pat. Nos. 5,814,618 and 5,789,156.

Any of the above-described promoters, or other known promoters, may be used to express siRNAs, miRNAs or amiRNAs of the invention. One skilled in the art is readily able to select a promoter as appropriate for a particular application.

The expression constructs and vectors of the invention may be used to prepare compositions for conferring traits to a target or host organism, as described herein below. In one aspect of the invention, such a composition is a nematicidal composition comprising a siRNA/miRNA comprising nucleotides 2-8 of SEQ ID NO: 1, 2, 12-23 and 39-40, for example, a siRNA/miRNA comprising the sequence set forth SEQ ID NO: 1, 2, 12-23 and 39-40. Compositions for conferring traits may also include two or more siRNAs/miRNAs.

Methods for Conferring Biotic and/or Abiotic Stress Tolerance

One aspect of the invention is a method for conferring abiotic and/or biotic stress tolerance to a plant, for example by increasing plant root growth, enhancing water optimization, enhancing pest tolerance, and/or increasing plant yield as compared to a control treatment or plant. In accordance with each method, a plant is contacted with a siRNA/miRNA of the invention. In one aspect, the contacting comprises expressing the siRNA/miRNA in a plant, as described herein below. In another aspect, the contacting refers to application of a formulation comprising the siRNA/miRNA to a plant or surrounding soil, also described herein below. Due to the variables in contacting the siRNA/miRNA with a plant, the desired traits do not necessarily occur, for example, by expression of the siRNA/miRNA in a plant. Rather, each of desired traits may occur independently of other desired traits, and must be selected for among plants that do not exhibit the desired trait.

A “host” or “host organism” as used herein refers to an organism that expresses a siRNA/miRNA. The host organism may transiently or stably express the siRNA/miRNA. A host organism may be a transgenic organism. A host organism may also comprise a target organism, i.e., a “target/host.”

The term “expression,” as used herein with regard to siRNA or miRNA refers to transcription of a siRNA/miRNA nucleotide sequence driven by its promoter. Expression as used herein also includes the production of siRNAs or miRNAs from larger RNA transcripts. As such, a host organism may express a RNA that is processed to produce or express one or more siRNAs or miRNAs.

Plants useful as target/host organisms include any of various photosynthetic, eukaryotic, multicellular organisms of the kingdom Plantae, including both monocots and dicots. The term “plant” includes reference to whole plants, plant parts, plant organs, plant tissues, plant cells, seeds, and progeny of the same. Plant cells include, without limitation, cells from seeds, suspension cultures, embryos, meristematic regions, callus tissue, leaves, roots, shoots, gametophytes, sporophytes, pollen, and microspores. Plant also refers to plants or plant parts that stably or transiently express a gene product, including a siRNA/miRNA.

Choosing a “control plant(s)” or “control treatment” is a routine part of an experimental setup and may include comparison to a corresponding wild type plant, comparison to a plant(s) that does not express a nucleic acid comprising a siRNA/miRNA, or comparison to a plant(s) not otherwise contacted with a siRNA/miRNA. A control plant is typically of the same plant species or even the same variety as the plant to be assessed. The control plant may also be a nullizygote of the plant to be assessed. Nullizygotes are individuals missing the transgene by segregation, i.e., a transgene expressing a nucleic acid comprising a siRNA/miRNA. A “control plant” as used herein refers not only to whole plants, but also to plant parts, including seeds and seed parts.

A “plant part” is any portion of a plant regardless of whether it is isolated or attached to an intact plant. The phrase “plant part” includes differentiated and undifferentiated tissues including, but not limited to the following: roots, stems, shoots, leaves, pollen, seeds, tumor tissue, and various forms of cells and culture (e.g., single cells, protoplasts, embryos, and callus tissue). The plant tissue may be in plant or in a plant organ, tissue, or cell culture. Plant part also includes plant products, such as grain, fruits, and nuts.

A “plant product” refers to an agricultural or commercial product created from a plant, plant part, or seed. Non-limiting examples of plant products include flowers, pollen, leaves, vines, stalks, fruits, vegetables, cucurbits, roots, tubers, cones, pods, seeds, beans, grains, kernels, and hulls.

Some plant products are processed and thus become “commodity products.” As used herein, “commodity products” include, but are not limited to, whole or processed seeds, beans, grains, kernels, hulls, meals, grits, flours, sugars, starches, protein concentrates, protein, lipids, carbohydrates, nucleic acids, metabolites, chlorophylls, waxes, oils, extracts, juices, concentrates, liquids, syrups, feed, silage, fiber, wood, pulp, paper, pigments, natural products, toxins, or other food or product produced from plants.

Commodity products containing one or more of the nucleotide sequences of the invention, or produced from a transformed plant, recombinant plant, or seed containing one or more of the nucleotide sequences of the invention are specifically contemplated as aspects of the invention as a means of identifying or detecting the source of the plant product or commodity. Such aspects are referred to herein as “biological samples.” The identification or detection of one or more of the nucleotide sequences of the invention in one or more biological samples is de facto evidence that the plant product or commodity product comprises a plant or plant part of the invention disclosed herein.

As used herein, a “nucleic acid from a plant,” “nucleic acid from plant part,” or “nucleic acid from plant product” comprising a nucleotide sequence of the invention, can be used to identify the plant or plant product using any number of techniques known in the art such as through PCR-based methods, southern blotting, northern blotting, or microarray analyses. In this particular aspect, the functionality of the target-specific siRNA/miRNA is immaterial and the presence of such nucleic acid in the plant or plant product serves to identify the plant source of a plant part or plant product.

The term “contacting” or phrase “contact with,” as used herein to describe bringing together siRNAs/miRNAs and a plant, means bringing together siRNA/miRNAs and nucleic acids of a plant so that they physically interact. The siRNA/miRNAs may be “contacted” or “administered” to the target in any manner that results in bringing into physical proximity a siRNA/miRNA and a target nucleic acid. In one aspect of the invention, a siRNA/miRNA may be transiently, stably, or inducibly expressed in a target/host organism, as described further herein below. In another aspect of the invention, a composition comprising a siRNA/miRNA may be sprayed onto the plant, or the siRNA/miRNA may be applied to soil in the vicinity of roots, taken up by the plant and/or target pest or pathogen, or a plant may be modified to express the siRNA/miRNA, also described further herein below.

As used herein, “contacting” also refers to placing a pest, pathogen, or target organism on or near a host plant, or part thereof, such that the pest, pathogen, or target organism has an opportunity to interact with, attack, or infect the plant or plant part, which effectively results in proximity between miRNAs expressed in the host plant and the target organism.

The siRNA/miRNA may be “contacted” or “administered” to the target in any manner that results in physical proximity of a siRNA/miRNA and a target nucleic acid permitting interaction. In one aspect of the invention, a siRNA/miRNA may be expressed within a host organism and then passively diffuse or be actively transported to a target organism. Expression within the host can be transient, or stable, and/or inducible. The siRNA/miRNA can be expressed as a precursor or inactive form that becomes active within the target organism. Expression in a host may be achieved using any of the expression constructs and vectors described herein.

Other examples of contacting include, but are not limited to, direct introduction into a cell (i.e., intracellularly); extracellular introduction into a cavity, interstitial space, or into the circulation of a target organism; oral introduction; the siRNA/miRNA may be introduced by bathing or soaking the target organism in a solution containing siRNA/miRNA. Methods for oral introduction include direct mixing of siRNA/miRNA with food of a target organism, as well as engineered approaches in which a species that is used as food is engineered to express a siRNA/miRNA, and then fed to the organism to be affected.

Where the target organism or host organism is a plant, a composition comprising a siRNA may be sprayed onto the plant, or the siRNA may be applied to soil in the vicinity of roots, taken up by the plant and/or target pest or pathogen, or a plant may be modified to express the siRNA.

For expression in a target/host, the siRNA/miRNA can be expressed as a precursor or inactive form that becomes active within the target organism. Expression in a target/host may be achieved using any of the expression constructs and vectors described herein.

A target/host organism expressing a heterologous siRNA/miRNA is “transgenic.” As used herein, the term “transgenic” refers to a host organism, or part or cell thereof, which comprises within its genome a heterologous polynucleotide. A transgenic host organism may be stably transformed or transiently transformed. If the heterologous siRNA/miRNA is stably integrated within the genome, it is passed on, or heritable, to successive generations. The heterologous siRNA/miRNA may be integrated into the genome alone or as part of an expression construct. Transgenic is used herein to include any cell, cell line, callus, tissue, plant part or plant, the genotype of which has been altered by the presence of a heterologous nucleic acid including those transgenics initially so altered as well as those created by breeding, sexual crosses, or asexual propagation from the initial transgenic cell.

Representative target/host plants include soybean (Glycine max), corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), tobacco (Nicotiana tabacum), potato (Solanum tuberosum), peanuts (Arachis hypogaea), cotton (Gossypium hirsutum), sweet potato (Ipomoea batatas), cassava (Manihot esculenta), coffee (Coffea ssp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus carica), guava (Psidium guajava), mango (Mangifera indica), olive (Olea europaea), papaya (Carica papaya), cashew (Anacardium occidental), macadamia (Macadamia integrifolia), almond (Prunus amygdalus), sugar beets (Beta vulgaris), oats, barley, vegetables, ornamentals, and conifers.

Additional target/host plants of the invention are crop plants, for example, cereals and pulses, maize, wheat, potatoes, tapioca, rice, sorghum, millet, cassaya, barley, pea, and other root, tuber, or seed crops. Important seed crops for the invention are oil-seed rape, sugar beet, maize, sunflower, soybean, and sorghum. Horticultural plants to which the invention may be applied may include lettuce, endive, and vegetable brassica including cabbage, broccoli, and cauliflower, and carnations, geraniums, petunias, and begonias. The invention may be applied to tobacco, cucurbits, carrot, strawberry, sunflower, tomato, pepper, chrysanthemum, poplar, eucalyptus, and pine. Optionally, plants of the invention include grain seeds, such as corn, wheat, barley, rice, sorghum, rye, etc. Optionally, plants of the invention include oil-seed plants. Oil seed plants include canola, cotton, soybean, safflower, sunflower, brassica, maize, alfalfa, palm, coconut, etc. Optionally, plants of the invention include leguminous plants. Leguminous plants include beans and peas. Beans include guar, locust bean, fenugreek, soybean, garden beans, cowpea, mung bean, lima bean, fava bean, lentils, chickpea, etc. Additional plants useful in the invention are row crops and broadcast crops. Non-limiting examples of useful row crops are corn, soybeans, cotton, amaranth, vegetables, rice, sorghum, wheat, milo, barley, sunflower, durum, and oats. Non-limiting examples of useful broadcast crops are sunflower, millet, rice, sorghum, wheat, milo, barley, durum, and oats. Target/host plants useful in the invention are monocots and dicots. Non-limiting examples of useful monocots are rice, corn, wheat, palm trees, turf grasses, barley, and oats. Non-limiting examples of useful dicots are soybean, cotton, alfalfa, canola, flax, tomato, sugar beet, sunflower, potato, tobacco, corn, wheat, rice, lettuce, celery, cucumber, carrot, and cauliflower, grape, and turf grasses. Target/host plants useful in the invention include plants cultivated for aesthetic or olfactory benefits. Non-limiting examples include flowering plants, trees, grasses, shade plants, and flowering and non-flowering ornamental plants. Target/host plants useful in the invention include plants cultivated for nutritional value, fibers, wood, and industrial products.

One skilled in the art will recognize the wide variety of target/host cells that can be transformed with the vectors disclosed herein. Non-limiting examples of such cells are those in embryogenic tissue, callus tissue types I, II, and III, hypocotyl, meristem, root tissue, tissues for expression in phloem, and the like.

Almost all plant tissues may be transformed during dedifferentiation using appropriate techniques described herein. Recipient cell targets include, but are not limited to, meristem cells, Type I, Type II, and Type III callus, immature embryos, and gametic cells such as microspores, pollen, sperm, and egg cells. It is contemplated that any cell from which a fertile plant may be regenerated is useful as a recipient cell. Type I, Type II, and Type III callus may be initiated from tissue sources including, but not limited to, immature embryos, immature inflorescences, seedling apical meristems, microspores, and the like.

Those cells that are capable of proliferating as callus also are recipient cells for genetic transformation. Techniques for transforming immature embryos and subsequent regeneration of fertile transgenic plants are well known in the art. Direct transformation of immature embryos obviates the need for long-term development of recipient cell cultures. Pollen, as well as its precursor cells, microspores, may be capable of functioning as recipient cells for genetic transformation, or as vectors to carry foreign DNA for incorporation during fertilization. Direct pollen transformation obviates the need for cell culture.

Meristematic cells (i.e., plant cells capable of continual cell division and characterized by an undifferentiated cytological appearance, normally found at growing points or tissues in plants such as root tips, stem apices, lateral buds, etc.) may represent another type of recipient plant cell. Because of their undifferentiated growth and capacity for organ differentiation and totipotency, a single transformed meristematic cell could be recovered as a completely transformed plant. In fact, it is proposed that embryogenic suspension cultures may be an in vitro meristematic cell system, retaining ability for continued cell division in an undifferentiated state, controlled by the media environment.

A wide variety of techniques are available for introducing siRNA/miRNAs of the invention into a target/host under conditions that allow for stable maintenance and expression of the siRNA/miRNA. The particular choice of a transformation technology will be determined by its efficiency to transform certain plant species as well as the experience and preference of the person practicing the invention with a particular methodology of choice. It will be apparent to the skilled person that the particular choice of a transformation system to introduce nucleic acid into plant cells is not essential to or a limitation of the invention, nor is the choice of technique for plant regeneration.

Transformation protocols as well as protocols for introducing heterologous nucleic acids into plants may vary depending on the type of plant or plant cell, i.e., monocot or dicot, targeted for transformation. Suitable methods of introducing the DNA construct include microinjection (Crossway et al. (1986) Biotechniques 4, 320-334; and U.S. Pat. No. 6,300,543); sexual crossing, electroporation (Riggs et al. (1986) Proc. Natl. Acad. Sci. USA 83, 5602-5606); Agrobacterium-mediated transformation (Townsend et al., U.S. Pat. No. 5,563,055 and 5,981,840); direct gene transfer (Paszkowski et al. (1984) EMBO J. 3, 2717-2722); and ballistic particle acceleration (see, e.g., Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6, 923-926). See also Weissinger et al. (1988) Ann. Rev. Genet. 22, 421-477; Sanford et al. (1987) Particulate Science and Technology 5, 27-37 (onion); Christou et al. (1988) Plant Physiol. 87, 671-674 (soybean); Finer and McMullen (1991) In Vitro Cell Dev. Biol. 27P, 175-182 (soybean); Singh et al. (1998) Theor. Appl. Genet. 96, 319-324 (soybean); Datta et al. (1990) Biotechnology 8, 736-740 (rice); Klein et al. (1988) Proc. Natl. Acad. Sci. USA 85, 4305-4309 (maize); Klein et al. (1988) Biotechnology 6, 559-563 (maize); Tomes, U.S. Pat. No. 5,240,855; Buising et al., U.S. Pat. Nos. 5,322,783 and 5,324,646; Klein et al. (1988) Plant Physiol. 91, 440-444 (maize); Fromm et al. (1990) Biotechnology 8, 833-839 (maize); Hooykaas-Van Slogteren et al. (1984) Nature 311, 763-764; Bowen et al., U.S. Pat. No. 5,736,369 (cereals); Bytebier et al. (1987) Proc. Natl. Acad. Sci. USA 84, 5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9, 415-418 and Kaeppler et al. (1992) Theor. Appl. Genet. 84, 560-566 (whisker-mediated transformation); D'Halluin et al. (1992) Plant Cell 4, 1495-1505 (electroporation); Li et al. (1993) Plant Cell Reports 12, 250-255 and Christou and Ford (1995) Annals of Botany 75, 407-413 (rice); Osjoda et al. (1996) Nature Biotechnology 14, 745-750 (maize via Agrobacterium tumefaciens); U.S. Pat. No. 5,736,369 (meristem transformation); and U.S. Pat. Nos. 5,302,523 and 5,464,765 (whiskers technology).

Nucleic acids of the invention may be introduced into plants by contacting plants with a virus or viral nucleic acids. Generally, such methods involve incorporating an expression construct of the invention within a viral DNA or RNA molecule. Further, it is recognized that useful promoters encompass promoters utilized for transcription by viral RNA polymerases. Methods for introducing expression constructs into plants and expressing a protein encoded therein, involving viral DNA or RNA molecules, are known in the art. See, e.g., U.S. Pat. Nos. 5,889,191; 5,889,190; 5,866,785; 5,589,367; and 5,316,931.

While various transformation methods are taught herein as separate methods, the skilled artisan will readily recognize that certain methods can be used in combination to enhance the efficiency of the transformation process. Non-limiting examples of such methods include bombardment with Agrobacterium-coated microparticles (EP486234) or microprojectile bombardment to induce wounding followed by co-cultivation with Agrobacterium (EP486233).

Direct delivery can also be used to transform hosts according to the invention. By way of non-limiting example, such direct delivery methods include polyethylene glycol treatment, electroporation, liposome mediated DNA uptake or the vortexing method. See, e.g., Freeman et al. (1984) Plant Cell Physiol. 29, 1353 and Kindle, (1990) Proc. Natl. Acad. Sci. USA 87, 1228. One form of direct DNA delivery is direct gene transfer into protoplasts from embryogenic cell suspension cultures. See Lazzeri and Lorz (1988) Advances in Cell Culture, Vol. 6, Academic Press, p. 291; OziasAkins and Lorz (1984) Trends in Biotechnology 2, 119.

The skilled artisan is aware of certain challenges of genotype-dependent transformation arising from low regeneration potential of cereals. Accordingly, in one embodiment of the invention, transformation is accomplished by a genotype-independent transformation approach based on the pollination pathway. Ohta (1986) Proc. Natl. Acad. Sci. USA 83, 715-719. In maize, high efficiency genetic transformation can be achieved by a mixture of pollen and exogenous DNA. Luo and Wu (1989) Plant Mol. Biol. Rep. 7, 69-77. Maize can be bred by both self-pollination and cross-pollination techniques. Maize has separate male and female flowers on the same plant, located on the tassel and the ear, respectively. Natural pollination occurs in maize when wind blows pollen from the tassels to the silks that protrude from the tops of the ears.

Transformation of tomato and melon with heterologous polynucleotides according to the invention can be accomplished into intact plants via pollination pathway. See Chesnokov, et al. (1999) USSR Patent No. 1708849; Bulletin of the USSR Patents, No. 4; Chesnokov and Korol (1993); Genetika USSR, 29, 1345-1355. The procedures of genetic transformation based on the pollination-fecundation pathway include: (i) employment of a mixture (paste) of the pollen and transforming DNA; (ii) delivery of the alien DNA into the pollen tube, after pollination; and (iii) microparticle bombardment of microspores or pollen grains.

In one aspect of the invention, target/host plants are transformed using Agrobacterium technology (e.g., A. tumefaciens and A. rhizogenes). Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, e.g., the methods described by Lloyd et al. (1986) Science 234, 464-466; Horsch et al. (1987) “Agrobacterium-mediated transformation of plants,” Plant Biology Alan R. Liss, NY pp 317-329; and Wang (2006) Agrobacterium protocols, Vol. 2, Humana Press, Totowa N.J. and U.S. Pat. No. 5,563,055.

Agrobacterium-mediated transformation can efficiently be used with dicotyledonous host plants of the invention including, by way of non-limiting example, Arabidopsis, corn, soybean, cotton, canola, tobacco, tomato, and potato.

Agrobacterium-mediated transformation is also applicable to nearly all monocotyledonous plants of the invention. By non-limiting example, such monocotyledonous plant technologies are adaptable to rice, wheat, and barley. See, e.g., Hiei et al. (1994) Plant J. 6, 271-282; Zhang et al. (1997) Mol. Biotechnol. 8, 223-231; Ishida et al. (1996) Nat. Biotechnol. 14, 745-750; McCormac et al. (1998) Euphytica 99, 17-25, Tingay S. et al. (1997) Plant J. 11, 1369-1376; and U.S. Pat. No. 5,591,616.

Agrobacterium-mediated transformation can be accomplished with cultured isolated protoplasts or by transformation of intact cells or tissues. Agrobacterium-mediated transformation in dicotyledons facilitates the delivery of larger pieces of heterologous nucleic acid as compared with other transformation methods such as particle bombardment, electroporation, polyethylene glycol-mediated transformation methods, and the like. In addition, Agrobacterium-mediated transformation appears to result in relatively few gene rearrangements and more typically results in the integration of low numbers of gene copies into the plant chromosome.

Modern Agrobacterium transformation vectors are capable of replication in E. coli as well as Agrobacterium, allowing for convenient manipulations as described. Klee et al. (1987) Ann. Rev. Plant Physiology 38, 467-486. Moreover, recent technological advances in vectors for Agrobacterium-mediated gene transfer have improved the arrangement of genes and restriction sites in the vectors to facilitate the construction of vectors capable of expressing various polypeptide-coding genes. The vectors described by Horsch et al. have convenient multi-linker regions flanked by a promoter and a polyadenylation site for direct expression of inserted polypeptide coding genes and are suitable for use according to the invention. Horsch et al. (1987) “Agrobacterium-mediated transformation of plants,” Plant Biology Alan R. Liss, NY pp 317-329. In addition, Agrobacterium containing both armed and disarmed Ti genes can be used for the transformations. In those plant strains where Agrobacterium-mediated transformation is efficient, it is the method of choice because of the facile and defined nature of the gene transfer.

When Agrobacteria are used to transform plant cells according to the invention, nucleic acids to be inserted can be cloned into special plasmids, namely either into an intermediate vector or into a binary vector. The intermediate vectors can be integrated into the Ti or Ri plasmid by homologous recombination owing to sequences that are homologous to sequences in the T-DNA. The Ti or Ri plasmid also comprises the vir region necessary for the transfer of the T-DNA. Intermediate vectors cannot replicate themselves in Agrobacteria. The intermediate vector can be transferred into Agrobacterium tumefaciens by means of a helper plasmid (conjugation).

Binary vectors can replicate themselves both in E. coli and in Agrobacteria. Such vectors can comprise a selection marker gene and a linker or polylinker, which are framed by the right and left T-DNA border regions. They can be transformed directly into Agrobacteria. Holsters et al. (1978) Mol. Gen. Genet. 163, 181-187. The Agrobacterium used as host cell can comprise a plasmid carrying a vir region. The vir region is necessary for the transfer of the T-DNA into the plant cell. Additional T-DNA may be contained. The bacterium so transformed is used for the transformation of plant cells. Plant explants can advantageously be cultivated with Agrobacterium tumefaciens or Agrobacterium rhizogenes for the transfer of the DNA into the plant cell. Whole plants can then be regenerated from the infected plant material (for example, pieces of leaf, segments of stalk, roots, but also protoplasts or suspension-cultivated cells) in a suitable medium, which may contain antibiotics or biocides for selection. The plants so obtained can then be tested for the presence of the inserted nucleic acids.

Following transformation, DNA constructs containing siRNA/miRNAs may be integrated of the into the target/host cell genome by homologous recombination, transposon-mediated insertion, or other methods of integration, including targeted integration at a particular host chromosomal site.

The cells from the plants that have stably incorporated the nucleotide sequence may be grown into plants in accordance with conventional techniques. See, e.g., McCormick et al. (1986) Plant Cell Reports 5, 81-84. These plants may then be grown, and either pollinated with the same transformed strain or different strains, and the resulting hybrid having constitutive expression of the desired phenotypic characteristic imparted by the nucleotide sequence of interest and/or the genetic markers contained within the target site or transfer cassette. Two or more generations may be grown to ensure that expression of the desired phenotypic characteristic is stably maintained and inherited, and then seeds are harvested to ensure expression of the desired phenotypic characteristic has been achieved.

Initial identification and selection of cells and/or plants comprising siRNA/miRNA expression constructs may be facilitated by the use of marker genes. Gene targeting can be performed without selection if there is a sensitive method for identifying recombinants, for example if the targeted gene modification can be easily detected by PCR analysis, or if it results in a certain phenotype. However, in most cases, identification of gene targeting events will be facilitated by the use of markers. Useful markers include positive and negative selectable markers as well as markers that facilitate screening, such as visual markers. Selectable markers include genes carrying resistance to an antibiotic such as spectinomycin, (e.g., the aada gene, Svab et al. (1990) Plant Mol. Biol. 14, 197); streptomycin, (Jones et al. (1987) Mol. Gen. Genet. 210, 86); kanamycin (e.g., nptII, Fraley et al. (1983) Proc. Natl. Acad. Sci. USA 80, 4803); hygromycin (e.g., HPT, Vanden Elzen et al. (1985) Plant Mol. Biol. 5, 299); gentamycin (Hayford et al. (1988) Plant Physiol. 86, 1216); phleomycin, zeocin, or bleomycin (Hille et al. (1986) Plant Mol. Biol. 7, 171); or resistance to a herbicide such as phosphinothricin (bar gene); or sulfonylurea (acetolactate synthase (ALS)) (Charest et al. (1990) Plant Cell Rep. 8, 643); genes that fulfill a growth requirement on an incomplete media such as HIS3, LEU2, URA3, LYS2, and TRP1 genes in yeast; and other such genes known in the art. Negative selectable markers include cytosine deaminase (codA) (Stougaard (1993) Plant J. 3, 755-761); tms2 (DePicker et al. (1988) Plant Cell Rep. 7, 63-66); nitrate reductase (Nussame et al. (1991) Plant J. 1, 267-274), SU1 (O'Keefe et al. (1994) Plant Physiol. 105, 473-482); aux-2 from the Ti plasmid of Agrobacterium; and thymidine kinase. Screenable markers include fluorescent proteins such as green fluorescent protein (GFP) (Chalfie et al. (1994) Science 263, 802; U.S. Pat. No. 6,146,826; U.S. Pat. No. 5,491,084; and PCT International Publication No. WO 97/41228); reporter enzymes such as 13-glucuronidase (GUS) (Jefferson R. A. (1987) Plant Mol. Biol. Rep. 5, 387, U.S. Pat. No. 5,599,670, and U.S. Pat. No. 5,432,081), 13-galactosidase (lacZ); alkaline phosphatase (AP); glutathione S-transferase (GST) and luciferase (U.S. Pat. No. 5,674,713; and Ow et al. (1986) Science 234, 856-859), visual markers like anthocyanins such as CRC (Ludwig et al. (1990) Science 247, 449-450) R gene family (e.g., Lc, P, S); A, C, R-nj, body and/or eye color genes in Drosophila, coat color genes in mammalian systems, and others known in the art.

One or more markers may be used in order to select and screen for targeting of a siRNA/miRNA to a particular genomic locus, which is also referred to as site-specific integration. One common strategy for site-specific integration involves using a promoterless selectable marker. Since the selectable marker lacks a promoter, random integration events generally do not lead to transcription of the gene. Gene targeting events will put the selectable marker under control of a promoter at the target site. Gene targeting events are identified by selection for expression of the selectable marker. Another common strategy utilizes a positive-negative selection scheme. This scheme utilizes two selectable markers, one that confers resistance (R⁺) coupled with one that confers sensitivity (S⁺), each with a promoter. When a heterologous nucleic acid containing the two markers is randomly inserted, the resulting phenotype is R⁺/S⁺. When a gene-targeting event is generated, the two markers are uncoupled and the resulting phenotype is R⁺/S⁻. Examples of using positive-negative selection are found in Thykjer et al. (1997) Plant Mol. Biol. 35, 523-530; and PCT International Publication No. WO 01/66717.

In another aspect of the invention, the step of contacting a plant with a siRNA/miRNA comprises applying or administering an effective amount of a composition comprising a siRNA/miRNA directly to the plant or surrounding growth media. In this instance, the effective concentration of a siRNA/miRNA composition is the concentration that would significantly increase root growth as compared to an untreated plant. Root growth includes increase of root number, depth, length, diameter, branching, girth, mass, surface area, formation of rhizobia or mycorrhiza nodules, or other relevant measure.

For example, a composition comprising the disclosed siRNA/miRNAs may be sprayed onto a plant surface as a liquid, semi-solid, or solid. In another aspect, a composition comprising the siRNA/miRNAs may be introduced into a root or stem by a physical means such as an injection.

For example, It is also anticipated that siRNA/miRNAs, amiRNAs, or constructs thereof may be formulated in a manner consistent with common agricultural practices and used as spray-on products. The formulations may include the appropriate stickers and wetters required for efficient foliar coverage as well as UV protectants to protect siRNAs, miRNAs, or constructs thereof from UV damage. Such additives are commonly used in the bioinsecticide industry and are well known to those skilled in the art. Such additives are commonly used in the bioinsecticide industry and are well known to those skilled in the art.

The siRNA/miRNA may also be expressed by microorganisms and the microorganisms may be applied onto a plant surface or introduced into a root, stem, leaf, or other plant part by a physical means such as an injection. In this instance, a bacterium engineered to produce and accumulate siRNA/miRNAs or miRNAs may be fermented and the products of the fermentation formulated as a spray-on product.

Another aspect of the invention also relates to a formulation comprising at least one siRNA, miRNA, or construct thereof and further, optionally at least one adjuvant and optionally, at least one surfactant. The effectiveness of the formulation may depend on the effectiveness of the spray application. Adjuvants can minimize or eliminate many spray application problems associated with the stability, solubility, incompatibility, suspension, foaming, drift, evaporation, volatilization, degradation, adherence, penetration, surface tension, and coverage of the siRNA, miRNA, or constructs thereof. Adjuvants are designed to perform specific functions, including wetting, spreading, sticking, reducing evaporation, reducing volatilization, buffering, emulsifying, dispersing, reducing spray drift, and reducing foaming. No single adjuvant can perform all these functions, but different compatible adjuvants often can be combined to perform multiple functions simultaneously. There are nonionic surfactants (no electrical charge), anionic surfactants (negative charge), and cationic surfactants (positive charge). The nature of the excipients and the physical form of the composition may vary depending upon the nature of the substrate that it is desired to treat. For example, the composition may be a liquid that is brushed or sprayed onto or imprinted into the material or substrate to be treated, or a coating or powder that is applied to the material or substrate to be treated.

Thus, in one aspect, the composition is in the form of a coating on a suitable surface, which adheres to the target/host organism. The spray containing the siRNA, miRNA, or constructs thereof is then internalized by the target/host, from where it can mediate RNA interference. The spray may be a pressurized/aerosolized spray or a pump spray. The spray may be dispersed through any method known to those having ordinary skill in the art. The particles may be of suitable size such that they adhere to the substrate to be treated or to a pest.

A composition comprising siRNAs of the invention can be applied before or after the crop is planted in the area of cultivation. Such applications can include an application of a siRNA composition, either alone or in combination with other agents, as described herein below. When a siRNA composition is used in combination with a second agent, it may be applied before, after, or concurrently with application of the second agent.

The forgoing methods for contacting a siRNA molecule of the invention with a plant may further include contacting the plant with a second agent to further enhance plant growth, as described herein below. Alternatively or in addition, plants contacted with a siRNA as disclosed herein can comprise a genetic background, either native or transgenic, that confers a desirable trait, also described herein below. In either instance, the combined use of a second agent or genetic background with a siRNA of the invention can elicit a synergistic effect, i.e., an effect that is greater than additive or greater than the effect of either agent/genetic background used alone. When assessing synergy, any relevant quantifiable index may be used, including indices with respect to individual plants (e.g., root mass) or indices with respect to an area of cultivation (e.g., yield). Useful indices include any one or more of those identified herein regarding abiotic stress resistance. One skilled in the art is readily able to identify and employ appropriate control plants and/or conditions to properly attribute an effect to an agent, genetic background, or combination of agents and/or genetic backgrounds. Synergy, expressed in terms of a “Synergy Index (SI),” generally can be determined by the method described by Kull et al., (1961) Applied Microbiology, 9, 538. See Colby (1967) “Calculating Synergistic and Antagonistic Responses of Herbicide Combinations,” Weeds, 15, 20-22.

In one aspect of the invention, the above-described second agents may include one or more siRNA/miRNA molecules and chemicals that promote biotic stress tolerance and/or abiotic stress resistance. For example, plants or compositions comprising siRNA/miRNAs of the invention may be used in combination with abscisic acid (ABA) or mimetics thereof, such as pyrabactin (see Peterson et al. (2010) Nature Structural & Molecular Biology 17, 1109-1113), β-aminobutyric acid (BABA) (see Jakab et al. (2005) Plant Physiol., 139, 267-274), salicylic acid (SA), and/or anthocyanin. The second agent may also comprise microorganisms that confer abiotic stress resistance, such as Fusarium spp. (see U.S. Patent Application Publication No. 20100227357).

In another aspect of the invention, plants or compositions comprising siRNA/miRNAs may be used in combination with additional abiotic stress resistance traits. For example, such a trait may include a heterolgous PYR/PYL receptor, optionally having increased sensitivity to ABA, such that inhibition of PP2C proteins occurs in response to lower amounts of ABA, or constitutive activity, such that inhibition of PP2C proteins occurs in the absence of ABA. In addition, expression of PP2C, such as HAB1, stimulates ABA binding to PYR/PYL receptors. See e.g., Melcher et al. (2009), Nature 462: 602-608; Fujii et al. (2009) Nature, 462: 660-664; and U.S. Patent Application Publication No. 20100216643, which are incorporated herein by reference in their entirety, and particularly with respect to compositions and methods for conferring drought tolerance.

Additional representative target/host plants having an abiotic stress resistance trait include plants with expression of a syntaxin (t-SNARE) protein (see U.S. Pat. No. 6,821,735, which is incorporated herein by reference in its entirety and particularly for its teaching of use of a syntaxin protein to mediate ABA signaling), AtCBF/DREB 1 (Kasuga et al. (1999) Nature Biotech. 17, 287-91); vacuolar pyrophosphatase, AVP1 (Gaxiola et al. (2001) Proc. Natl. Acad. Sci. 98, 11444-19 and U.S. Pat. No. 7,534,933); ORF69 (see U.S. Pat. No. 7,847,154); OsNACx (see U.S. Pat. No. 7,834,244); ferrochelatase (see U.S. Pat. No. 7,812,223); PGR5 (see U.S. Pat. No. 7,807,875); DRO2 (see U.S. Pat. Nos. 7,795,499 and 7,432,416); DRO3 (see U.S. Pat. No. 7,754,945); NADP-malic enzyme (see Laporte et al. (2002) J. Exp. Bot. 53:699-705); 9-cis-epoxycarotenoid dioxygenase (NCED) (see Qin et al. (2002) Plant Physiol. 128, 544-51; luchi et al. (2001) Plant J. 27, 325-33); trehalose-6-phosphate synthase (see Yeo et al. (2000) Mol. Cell. 10:263-8 and U.S. Patent Application Publication No. 20080138903); trehalose-6-phosphate phosphatase (see U.S. Patent Application Publication No. 20100205692); BiP (see Alvim et al. (2001) Plant Physiol. 126, 1042); ABF3/ABF4 (see Kang et al. (2002) Plant Cell 14, 343-357 and U.S. Patent Application Publication No. 20030204874); a cold shock protein (Csp), such as cspB (see U.S. Pat. No. 7,786,353); poly(ADP-ribose) polymerase (PARP) (see U.S. Pat. No. 7,786,349); PRDT1 (see U.S. Pat. No. 7,786,346); a flavin-containing monooxygenase, such as YUCCA6 (see U.S. Pat. No. 7,763,772); Glutathione-S transferase, such as PjGST from Prosopis juhflora (see U.S. Pat. No. 7,655,837); OSISAP1 (see U.S. Pat. No. 7,576,263); MAPK5 (see U.S. Pat. No. 7,345,219); cytokinin oxidase (see U.S. Pat. No. 7,332,316); a CBF gene (for C-repeat/DRE binding factor) (see U.S. Pat. No. 7,317,141); HVA22/HVA1 (see U.S. Pat. No. 6,951,971); Snf1-related protein kinase (SnRK) (see U.S. Patent Application Publication No. 20100281574); PRDT1 (see U.S. Patent Application Publication No. 20100275321); a NAC transcriptional activator, such as NAC3 and NAC4 (see U.S. Patent Application Publication No. 20100223695); a NADP-specific glutamate dehydrogenase enzyme, such as that encoded by gdhA (see U.S. Patent Application Publication No. 20100223693); SNAC2 (see U.S. Patent Application Publication No. 20100186108); spermidine synthase (see U.S. Patent Application Publication No. 20100083401); DREB/CBF (see Hussain et al. (2010) published online at internet address dx.doi.org/10.1002/btpr.514); ALDH7 antiquitin-like protein GmTP55 (Rodrigues et al. (2006) J. Exp. Botany 57, 1909-18, a molybdenum cofactor sulfurase (see U.S. Patent Application Publication No. 20080184395), a farnesyl transferase (see U.S. Patent Application Publication No. 20090293156), and/or Am244 (see U.S. Patent Application Publication No. 20090313726). See also U.S. Pat. Nos. 7,763,775; 7,718,788; 7,427,697; and 7,388,125; and U.S. Patent Application Publication Nos. 20100175145 and 20100005546. In the foregoing examples, elevated expression may include transformation of a plant with a heterologous nucleic acid encoding the gene to be overexpressed or overexpression of an endogenous gene, as appropriate.

Additional representative target/host plants are plants having genetic backgrounds with improved abiotic tolerance including naturally occurring or hybrid strains. For example, the soybean variety Jackson shows high water utilization efficiency. In addition, soybean cultivars Lee-74 and Wright had higher dry matter accumulation, greater height, and better germination under stress conditions (see Kpoghomou et al. (2008) Journal of Agronomy and Crop Science 164, 153-159). In maize, hybrids marketed as containing AGRISURE ARTESIAN™ technology show improved water utilization. As another example, wheat strains showing improved drought tolerance include Sahel 1, Giza 168, Rufom-5 (see Bayoumi et al. (2008) Af. J. Biotechnol. 7, 2341-2352).

In addition to the foregoing disclosure of additional agents/traits/genetic backgrounds for conferring abiotic stress resistance, in other aspects of the invention, target/host plants may show improved resistance to pests/pathogens such as viruses, nematodes, insects or fungi, and the like (see e.g., U.S. Pat. No. 7,875,429, which is incorporated herein by reference in its entirety, and with particularly for its teaching of an invertase transgene that confers insect resistance in maize); traits desirable for animal feed such as high oil content, balanced amino acid content, increased digestibility, etc.; traits desirable for processing, such as modified oils and starches; agronomic traits such as male sterility, stalk strength, flowering time; traits affecting plant morphology (see e.g. U.S. Pat. No. 7,588,939, which is incorporated herein by reference in its entirety and particularly for its teaching of use of RAMOSA genes for altering plant morphology and improving plant yield) and/or or transformation technology traits such as cell cycle regulation or gene targeting.

Where a siRNA/miRNA of the invention is expressed in a target/host, which is used in combination with a target/host having a genetic background or trait conferring a desirable phenotype, as described above, the above-described stacked combinations can be created by any method including, but not limited to, breeding plants by any conventional or TopCross methodology, or genetic transformation. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, e.g., PCT International Publication Nos. WO 99/25821, WO 99/25854, WO 99/25840, WO 99/25855, and WO 99/25853.

### EXAMPLES

The following examples are presented only for the purpose of illustration and description and are not intended to be exhaustive or to limit the invention to the precise forms disclosed. Numerous modifications and adaptations thereof will be apparent to those skilled in the art without departing from the spirit and scope of the invention.

### Example 1

### siRNA Library Design and Construction

A 21-nucleotide small interfering RNA library was designed with a randomized seed sequence located at positions 2-8 from the 5′-end, and positions 1, 9-21 were fixed. Uridine residues were chosen for positions 20 and 21 in order to increase the stability of the molecule for in vitro screening. The non-seed sequence generated from the consensus C. elegans miRNA is 5′-UNNNNNNNUGUUGAUCUGGUU-3′, (SEQ ID NO: 9) where N indicates a random nucleotide (i.e., either A, C, G, or U) in the seed sequence. The non-seed sequence or nucleic acid also has partial complementarity to the soybean ETR1 mRNA. A siRNA library of this exemplary sequence consists of 4⁷ (i.e., 4×4×4×4×4×4×4) different RNA molecules, or 16,384 possible sequences.

In order to reduce the complexity of an RNA library (i.e., the number of sequences contained in the library), a subset of sequences were excluded from the library. In particular, the complexity of the siRNA library was reduced by computationally excluding nucleotides that occurred at a particular position in known miRNA seed sequences at lower frequencies. In addition, small RNA sequences were excluded from the library if they contained homonulceotide quadruplets, such as AAAA. Further, sequences having a GC-content in positions 1-9 (i.e., position 1 and the seed sequence) greater than the GC-content of positions 11-19 were also excluded. After these additional parameters were considered, the number of siRNA sequences in the library was reduced to 563 sequences.

The 563 siRNAs were synthesized as duplexes using standard automated synthesis. In order to enhance duplex stability, the 3′-residues may be stabilized against nucleolytic degradation, e.g., they consist of purine nucleotides. Alternatively, substitution of pyrimidine nucleotides by modified analogues, e.g., substitution of uridine by 2′-deoxythymidine is tolerated and does not affect the efficiency of RNA interference. siRNAs were synthesized with a dTdT dinucleotide at the 3′-end as an overhang to increase duplex stability and prevent nucleolytic degradation.

The following siRNAs were considered for in vivo tests:

TABLE 1
siRNA sequences

| | siRNA Sequence | SEQ ID |
|---|---|---|
| siRNA ID | (5′→3′) | NO: |
| siRNA0097 | UAAGUUCAUGUUGAUCUGGUU | 1 |
| siRNA0145 | UAGCUUGAUGUUGAUCUGGUU | 2 |
| siRNA0043 | UAACUUAUUGUUGAUCUGGUU | 12 |
| siRNA0046 | UAACUUCUUGUUGAUCUGGUU | 13 |
| siRNA0192 | UAGGUUGGUGUUGAUCUGGUU | 14 |
| siRNA0243 | UAUCUUCGUGUUGAUCUGGUU | 15 |
| siRNA0309 | UGACAGGAUGUUGAUCUGGUU | 16 |
| siRNA0382 | UGAGGUCAUGUUGAUCUGGUU | 17 |
| siRNA0423 | UGGUAUGGUGUUGAUCUGGUU | 18 |
| siRNA0458 | UGGGAUCUUGUUGAUCUGGUU | 19 |
| siRNA0483 | UGUCAGAUUGUUGAUCUGGUU | 20 |
| siRNA0514 | UGUCGUGAUGUUGAUCUGGUU | 21 |
| siRNA0531 | UGUCUUCGUGUUGAUCUGGUU | 22 |
| siRNA0569 | UGUGUGAUUGUUGAUCUGGUU | 23 |

All the siRNAs described in Table 1 have identical non-seed sequences (nucleotides 1 and 9-21) but variable seed sequences (nucleotides 2-8). The seed sequences have at least partial complementarity to a portion of a soybean ETR1 nucleic acid. The siRNA0097 seed sequence has full complementarity to a portion of the soybean ETR1 nucleic acid. The siRNAs siRNA0097 and siRNA0145 can create duplexes within an amiRNA that comprise a siRNA/siRNA* duplex that can interfere with the mRNA target, as well as, the complementary miRNA target or miRNA*. In recent years, it has been discovered that the miRNA* strand of some of the miRNA/miRNA* duplexes could also be loaded into the RISC and interfere with the expression of its complementary mRNA target (Kulcheski, et al. (2011)BMC Genomics 12:307). Therefore it is possible that the miRNA* or siRNA* can be loaded into the RISC and used to silence a target mRNA. The miRNA0097* and the miRNA0145* produce miRNAs that can form complementary binding with the gma-ETR1 mRNA (see below), however, the binding site is different than the binding site of the miRNA0097 and miRNA0145. Therefore it is possible that the miRNAs produced by miRNA0097* and miRNA0145* can down-regulate the expression of the gma-ETR1 gene, as well as the amiRNA0097 and amiRNA0145.

The following is an example of potential binding sites for miRNA0097 produced from the amiRNA0097 (SEQ ID NO: 3) and miRNA0145 produced from the amiRNA0145 (SEQ ID NO: 4) on the gma-ETR1 mRNA.

Binding Between miRNA0097 or miRNA0145 and gma-ETR1 mRNA

(miRNA produced amiRNAs found in SEQ ID NOS: 3 & 4 and mRNA produced from nucleotides 1761-1780 of the gma-ETR1 full CDS, SEQ ID NO: 36)

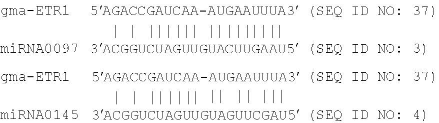

Binding Between miRNA0097* or miRNA0145* and gma-ETR1 mRNA

(mRNA produced from nucleotides 1635-1654 of gma-ETR1 (SEQ ID NO: 36)

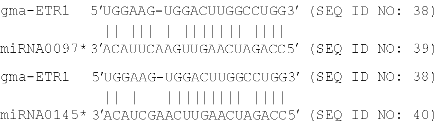

As described above, the miRNA0097/miRNA0097* and miRNA0145/miRNA0145* are capable of binding to the soybean ETR1 nucleic acid. Therefore, it is possible that alone or in combination miRNA0097 and miRNA0097* or miRNA0145 and miRNA0145* can modulate the expression of the soybean ETR1 nucleic acid.

The miRNA0043 or miRNA0046 sequence produced from amiRNA0043 (SEQ ID NO: 24) and amiRNA0046 (SEQ ID NO: 25) shows low overall complementarity to a soybean ETR1 nucleic acid, especially in the seed sequence, as well as, gaps in complementarity that would prevent proper base paring with gma-ETR1, as seen below.

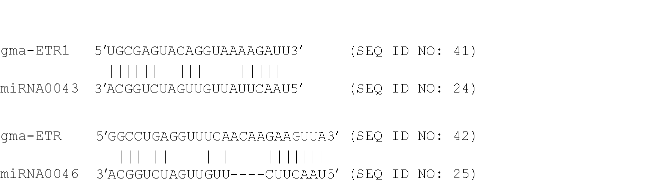

### Example 2

### Construction of amiRNAs

The design of microRNAs (miRNA) for expression in plant host cells essentially follows the literature of Schwab et al, where miRNAs were designed to target individual genes or groups of endogenous genes in a plant cell. See Schwab et al. (2006) Plant Cell 18, 1121-1133 and Alvarez et al. (2006) Plant Cell 18, 1134-1151. The 15 siRNA molecules were assembled into artificial microRNA (amiRNA) constructs. Soybean microRNA precursor, gma-MIR164, was used as the backbone of the amiRNA. The miR164/miR164* sequence on this precursor was replaced by siRNA/siRNA* sequence, while the mismatch positions on the miR164/miR164* duplex were maintained in the artificial siRNA/siRNA* sequence by making mutations on the siRNA* passenger strand. Sequences of the amiRNAs are shown in Table 2. Each miRNA was inserted into a gma-aMIR164 precursor and overexpressed as a miRNA driven by the root preferred promoter CMP (Stavolone et al. (2003) Journal of General Virology 84, 3459-3464) in soybean hairy roots. The base binary vector is 15312, which has a reporter gene expression cassette of prCMP:cZsGreen:tNOS. Vectors used in the transgenic amiRNA expression experiments are provided in Table 3.

TABLE 2
amiRNA sequences

| amiRNA ID | amiRNA Sequence (5′→3′) | SEQ ID NO: |
|---|---|---|
| amiRNA0097 | AGCTCCTTGTTAAGTTCATGTTGATCTGGCAAGTCTCTTGGATC | |
| | TCAAATGCCACTGAACCCTTTGCCAGATCAAGTTGAACTTACAA | 3 |
| | CACGGGTTT | |
| amiRNA0145 | AGCTCCTTGTTAGCTTGATGTTGATCTGGCAAGTCTCTTGGATC | |
| | TCAAATGCCACTGAACCCTTTGCCAGATCAAGTTCAAGCTACAA | 4 |
| | CACGGGTTT | |
| amiRNA0043 | AGCTCCTTGTTAACTTATTGTTGATCTGGCAAGTCTCTTGGATC | |
| | TCAAATGCCACTGAACCCTTTGCCAGATCAAGTATAAGTTACAA | 24 |
| | CACGGGTTT | |
| amiRNA0046 | AGCTCCTTGTTAACTTCTTGTTGATCTGGCAAGTCTCTTGGATC | |
| | TCAAATGCCACTGAACCCTTTGCCAGATCAAGTAGAAGTTACAA | 25 |
| | CACGGGTTT | |
| amiRNA0192 | AGCTCCTTGTTAGGTTGGTGTTGATCTGGCAAGTCTCTTGGATC | |
| | TCAAATGCCACTGAACCCTTTGCCAGATCAAGTCCAACCTACAA | 26 |
| | CACGGGTTT | |
| amiRNA0243 | AGCTCCTTGTTATCTTCGTGTTGATCTGGCAAGTCTCTTGGATC | |
| | TCAAATGCCACTGAACCCTTTGCCAGATCAAGTCGAAGATACAA | 27 |
| | CACGGGTTT | |
| amiRNA0309 | AGCTCCTTGTTGACAGGATGTTGATCTGGCAAGTCTCTTGGATC | |
| | TCAAATGCCACTGAACCCTTTGCCAGATCAAGTTCCTGTCACAA | 28 |
| | CACGGGTTT | |
| amiRNA0382 | AGCTCCTTGTTGAGGTCATGTTGATCTGGCAAGTCTCTTGGATC | |
| | TCAAATGCCACTGAACCCTTTGCCAGATCAAGTTGACCTCACAA | 29 |
| | CACGGGTTT | |
| amiRNA0423 | AGCTCCTTGTTGGTATGGTGTTGATCTGGCAAGTCTCTTGGATC | |
| | TCAAATGCCACTGAACCCTTTGCCAGATCAAGTCCATACCACAA | 30 |
| | CACGGGTTT | |
| amiRNA0458 | AGCTCCTTGTTGGGATCTTGTTGATCTGGCAAGTCTCTTGGATC | |
| | TCAAATGCCACTGAACCCTTTGCCAGATCAACAAGATCCCACAA | 31 |
| | CACGGGTTT | |
| amiRNA0483 | AGCTCCTTGTTGTCAGATTGTTGATCTGGCAAGTCTCTTGGATC | |
| | TCAAATGCCACTGAACCCTTTGCCAGATCAAGTATCTGACACAA | 32 |
| | CACGGGTTT | |
| amiRNA0514 | AGCTCCTTGTTGTCGTGATGTTGATCTGGCAAGTCTCTTGGATC | |
| | TCAAATGCCACTGAACCCTTTGCCAGATCAAGTTCACGACACAA | 33 |
| | CACGGGTTT | |
| amiRNA0531 | AGCTCCTTGTTGTCTTCGTGTTGATCTGGCAAGTCTCTTGGATC | |
| | TCAAATGCCACTGAACCCTTTGCCAGATCAAGTCGAAGACACAA | 34 |
| | CACGGGTTT | |
| amiRNA0569 | AGCTCCTTGTTGTGTGATTGTTGATCTGGCAAGTCTCTTGGATC | |
| | TCAAATGCCACTGAACCCTTTGCCAGATCAACAATCACACACAA | 35 |
| | CACGGGTTT | |

TABLE 3
Vectors Used in Transgenic amiRNA Expression Experiment

| amiRNA ID | Vector ID | SEQ ID NO |
|---|---|---|
| amiRNA0097 | pKS100 | 5 |
| amiRNA0145 | pKS101 | 6 |
| Vector Control | 15312-prCMP-GUS-tNOS | 7 |
| Vector Control | Empty 15312 | 8 |

### Example 3

### In Vivo Transgenic Root Assays

The binary vectors described above were transformed into Agrobacterium rhizogenes Strain K599 cells. The Agrobacterium harboring these vectors were inoculated onto soybean cotyledons to induce transgenic hairy roots. An equal number of cotyledons (120) were inoculated with each strain. The cotyledons were placed on co-cultivation media for 6 days, and then transferred onto selection media. One week later (13 Days After Inoculation, “DAI”), the number of cotyledons with transgenic hairy roots within each group of 120 cotyledons was counted. Another week later (20 DAI), the number of cotyledons with transgenic hairy roots within each group of 120 cotyledons were counted. At each time point, the total numbers of transgenic roots within each group of 120 cotyledons were counted as well. Transgenic roots were identified by the green fluorescence due to the expression of the ZsGreen reporter gene. When the amiRNAs described in Table 4 were assayed only amiRNA0097 and amiRNA0145 showed increase roots over the control constructs. The other amiRNA vector constructs gave results similar to control constructs. The results of this experiment are shown in Table 4.

TABLE 4
Transgenic amiRNA Expressions Results

| | | | | | | Number of | |
|---|---|---|---|---|---|---|---|
| | | Number of | | Number of | | Cotyledons | |
| | Number of | Fluorescent | | Cotyledons with | | without | |
| | Inoculated | Roots | | Fluorescent Roots | | Fluorescent Roots | |
| Construct | Cotyledons | 13 DAI | 20 DAI | 13 DAI | 20 DAI | 13 DAI | 20 DAI |
| pKS100 (amiRNA0097) | 120 | 23 | 464 | 15 | 120 | 105 | 3 |
| pKS101 (amiRNA0145) | 120 | 36 | 654 | 23 | 120 | 97 | 0 |
| 15312-prCMP-GUS-tNOS | 120 | 9 | 109 | 4 | 114 | 116 | 6 |
| 15312 | 120 | 11 | 124 | 5 | 108 | 115 | 12 |

Chi-square Tests were performed to determine whether the increased number of rooting cotyledons were statistically significant compared to the control experiments. The Chi-square analyses indicated that the insertion of GUS expression cassette in the vector 15312 prCMP-GUS-tNOS did not significantly increase the numbers of root cotyledons, compared to the empty vector control 15312 (p>0.5 at 13 DAI and p>0.05 at 20 DAI). In contrast, insertion of the amiR0097 or amiR0145 expression cassettes significantly increased the numbers of root cotyledons, compared to the empty vector control 15312 (p<0.001 for both amiRNAs at both 13 DAI and 20 DAI), and the insert control, 15312 prCMP-GUS-tNOS (p<0.001 for both amiRNAs at 13 DAI, and p<0.05 for both amiRNAs at 20 DAI). Moreover, the amiR145 expression cassette induced more transgenic roots than amiR0097 at 13 DAI (p<0.05).

These experiments indicate that only the amiR0097 and amiR0145 promote root growth and changes in root architecture in soybean hairy roots and may be used to promote tolerance against abiotic environmental stressors such as drought.

### Example 4

### Transgenic Plant Assays

Agrobacterium containing amiRNA0097 and amiRNA0145 as described above were used to transform soybean. Transformation of soybean to produce transgenic soybean plants was accomplished using immature seed targets of variety Williams 82 via A. tumefaciens-mediated transformation. Explant materials and media recipes were essentially as described in Hwang et al. (PCT International Publication No. WO 08/112,044) and Que et al. (PCT International Publication No. WO 08/112,267), with some variations as noted below. Using this method, genetic elements within the left and right border regions of the transformation plasmid are efficiently transferred and integrated into the genome of the plant cell, while genetic elements outside these border regions are generally not transferred.

Maturing soybean pods were harvested from greenhouse-grown plants, sterilized with diluted bleach solution, and rinsed with sterile water. Immature seeds were then excised from seedpods and rinsed briefly with sterile water. Explants were prepared from sterilized immature seeds as described in Hwang et al. (PCT International Publication No. WO 08/112,044) and infected with A. tumefaciens strain EHA101 harboring the transformation binary vector 18963 and allowed to incubate for an additional 30 to 240 minutes. Excess A. tumefaciens suspension was removed by aspiration and the explants were moved to plates containing a non-selective co-culture medium. The explants were co-cultured with the remaining A. tumefaciens at 23° C. for 4 days in the dark and then transferred to recovery and regeneration medium supplemented with an antibiotics mixture consisting of ticarcillin (75 mg/L), cefotaxime (75 mg/L) and vancomycin (75 mg/L) where they were incubated in the dark for seven days.

The explants were then transferred to regeneration medium containing hygromycin B (3 to 6 mg/L) and a mixture of antibiotics consisting of ticarcillin (75 mg/L), cefotaxime (75 mg/L) and vancomycin (75 mg/L) to inhibit and kill A. tumefaciens. Shoot elongation and regeneration was carried out in elongation media containing 2-4 mg/L of hygromycin B. The hygromycin phosphor-transferase (HPT) gene was used as a selectable marker during the transformation process. Regenerated plantlets were transplanted in soil as described (PCT International Publication No. WO 08/112,267) and tested for the presence of HPT and CMP promoter sequences using TaqMan PCR analyses. Ingham et al. (2001) Biotech 31, 132-140. This screen allows for the selection of transgenic events that carry the T-DNA and are free of vector DNA. Plants positive for HPT gene and CMP sequences and negative for the spectinomycin (spec) gene were transferred to the greenhouse for analysis of miRNA expression and seed setting.

When the roots were about 2-3 inches, they were then transplanted into 1-gallon pots using Fafard #3 soil and 30 grams of incorporated Osmocote Plus 15-9-12. They were watered thoroughly and placed in the cubicle under florescent lighting set to a 16-hour day. The temperatures are 85° F. (29.4° C.) during the day and 70° F. (21° C.) at night. Plants were watered once daily.

The plants remain in the cubicle until secondary Taqman sampling has been performed, typically 1-2 weeks. The plants were then placed on an automatic drip watering system and watered twice daily. A cage is placed over the plant, and it may be pruned very lightly if needed. The lighting is a combination of Metal Halide and Sodium Vapor fixtures with 400- and 1000-watt bulbs with a 10-hour day period. The outside wall is darkened to keep out light that would extend the day length. Temperatures are set at 79° F. (26° C.) during the day and 70° F. (21° C.) at night. The humidity is ambient.

The plants are maintained in this manner until pods reach maturity, approximately 100 days based on the date of the Taqman selection. The pods are then harvested, placed in a paper bag, air-dried for 2-days, and then machine dried at 80° F. (27° C.) for 2-additional days. The pods are shelled and the T1 seeds are harvested and stored at 4° C. until further testing.

Analysis of the expression of gma-ETR1 in transgenic soybean expression amiRNA0097 and amiRNA0145 was accomplished using a qRT-PCR assay. The leaves of the transgenic soybean events were sampled with a qRT-PCR assay to determine the expression level of ETR1 in these plants. The results shown in Table 5 indicate that in both events, the expression of the gma-ETR1 was down regulated. In amiRNA0097 events, the down-regulation of gma-ETR1 was more than that in amiRNA0145 events. This can be explained by the better binding of the miRNA0097 and miRNA0097* to the gma-ETR1 than the miR0145 and miRNA0145* respectively (see above).

TABLE 5

| Construct | Wild Type | amiRNA0097 | amiRNA0145 |
|---|---|---|---|
| Mean Relative | 33.96 | 22.42 | 11.80 |
| Expression Level | | | |
| n | 9 | 14 | 21 |
| Standard Error | 5.02 | 4.30 | 1.40 |

The transgenic soybean plants described above, showed no negative phenotypes and appeared to be normal in appearance and characteristics when compared to the control plants.

### Example 5

### Determining Root Characteristics

Transgenic seeds grown in a greenhouse or field as described previously can be evaluated for root characteristics by various methods. In one method, described in Ortiz-Ribbing et al. (2003) Crop Management, entitled “Evaluation of Digital Image Acquisition Methods for Determining Soybean Root Characteristics” and published online, the soybean plants are grown in the field, root and soil are sampled to a depth of 10 to 12 inches, and soaked in 5% sodium hexametaphosphate solution to remove the soil. Root volumes are measured by the water displacement methods, as well as, digitally scanned to determine root characteristics. Alternatively, seedling root assays can be performed by germinating seeds in media as described in Negi, et al., (2008) The Plant Journal 55, 175-187.

### Example 6

### Screening for Pod Retention

Transgenic seeds are planted in the field in 12-foot rows with 8 seeds per foot and 30 inches between rows. Plots are laid out in a randomized, replicated design. At reproductive stages R3, R4, R5, or maturity, 8 plants per row are harvested for pod mapping. Number of branches including main stem, number of nodes, and number of pods per node are recorded for each plant within a row. Pod number and distribution are scored in comparison to control plants grown within the replica. Results across replicas and locations are combined. Plants are sampled and tested for the presence of HPT and CMP promoter sequences using TaqMan PCR analyses, as described above. The number of pods per node is compared between transgenic plants and control plants.

### Example 6

### Screening for Increased Yield

Seeds are planted in the field in 12-foot rows with 8 seeds per foot and 30 inches between rows. Plots are laid out in a randomized, replicated design. Each row is harvested separately. Pounds of seeds per plot and moisture are determined. Pounds per seeds per plot are corrected to 13% moisture and are scored in comparison to control plants grown within the replica. Results for replicates within a location and across several locations are combined.

Seeds derived from pooled plants from a single row will be planted in the field as described above. At maturity, the number of pods per node may be re-determined to confirm results obtained in the previous generation. Similarly, the derived seed may be re-screened to confirm stability of the HPT and CMP promoter sequences. As the number of pods per node is one component of yield, it is expected that yield will be increased in the lines displaying reduction in expression of ETR1.

What is claimed is:

1. A method for preparing a transgenic plant having a normal germination phenotype as compared to a control plant comprising:
a) expressing in a plant a nucleic acid comprising a miRNA comprising a seed and a non-seed nucleic acid and having at least partial complementarity to a target nucleic acid, wherein the target nucleic acid comprises an ethylene response nucleic acid; and
b) growing the trangenic plant having a normal germination phenotype.

2. The method of claim 1, wherein the seed nucleic acid is only partially complementary to the target nucleic acid.

3. The method of claim 2, wherein the seed nucleic acid is fully complementary to a target nucleic acid.

4. The method of any one of claims 1-3, wherein the target nucleic acid is a ETR1 nucleic acid.

5. The method of any one of claims 1-4, wherein the target nucleic acid is from Glycine max.

6. The method of any one of claims 1-5, wherein the target nucleic acid is SEQ ID NO: 36.

7. The method of any one of claims 1-6, wherein the miRNA comprises SEQ ID NO: 1 or SEQ ID NO: 2.

8. The method of any one of claims 1-7, wherein the miRNA is contained within an amiRNA.

9. The method of claim 8, wherein the amiRNA comprises a gma-MIR164 backbone.

10. The method of claim 9, wherein the amiRNA comprises SEQ ID NO: 3 or SEQ ID NO: 4.

11. The method of any one of claims 10-12, wherein the amiRNA is operably linked to a tissue preferred promoter.

12. The method of claim 13, wherein the tissue preferred promoter is a root preferred promoter.

13. The method of claim 14, wherein the root preferred promoter is a CMP promoter.

14. The method of claim 1, wherein the seed nucleic acid comprises nucleotides occupying positions 2-8 of the miRNA and wherein the non-seed nucleic acid comprises nucleotides occupying positions 1 and 9-21 of the miRNA.

15. The method of claim 11, wherein 5 nucleotides of the seed nucleic acid are complementary to the target nucleic acid and are able to properly base pair with the target mRNA.

16. The method of claim 12, wherein 6 nucleotides of the seed nucleic acid are complementary to the target nucleic acid and are able to properly base pair with the target mRNA.

17. The method of claim 13, wherein 7 nucleotides of the seed nucleic acid are complementary to the target nucleic acid and are able to properly base pair with the target mRNA.

18. The method of any one of claims 1-11, wherein the amiRNA is capable of producing two miRNAs that are at least partially complementary to a ethylene response mRNA.

19. A plant produced by the method of any one of claims 1-21.

20. A method of improving root growth in a plant while maintaining normal germination comprising
a) transforming a plant cell with a vector comprising an amiRNA expressing a miRNA has at least partial complementarity to a plant ETR mRNA; and
b) regenerating a plant with improved root growth from the plant cell.

21. The method of any one of claim 22, wherein the amiRNA is operably linked to a tissue preferred promoter.

22. The method of claim 22, wherein the tissue preferred promoter is a root preferred promoter.

23. The method of claim 22, wherein the root preferred promoter is a CMP promoter.

24. A plant produced by the method of any one of claims 22-25

25. Use of an miRNA with at least partial complementarity to a plant ETR mRNA in a plant for improved root growth while maintaining normal germination.

26. The use of claim 27, wherein the miRNA is expressed in the plant with a tissue preferred promoter.
